(19) United States
(12) Patent Application Publication
(10) Pub. No.: US 2023/0377693 A1
(43) Pub. Date: Nov. 23, 2023

(54) HYBRID QUANTUM-CLASSICAL COMPUTING SIMULATION OF CHEMICAL SYSTEMS

(71) Applicant: Cambridge Quantum Computing Limited, London (UK)

(72) Inventor: Michal Piotr Krompiec, Cambridge (GB)

(21) Appl. No.: 18/152,971

(22) Filed: Jan. 11, 2023

Related U.S. Application Data
(63) Continuation-in-part of application No. 17/931,616, filed on Sep. 13, 2022.
(60) Provisional application No. 63/344,592, filed on May 22, 2022.

(51) Int. Cl. G16C 10/00; G06N 10/20

(52) U.S. Cl. CPC G16C 10/00; G06N 10/20

(57) ABSTRACT

A chemical system is simulated using a hybrid quantum-classical computing system. The classical component of the system evaluates class selection metrics based on a structure of the chemical system; determines whether the class selection metrics satisfy one or more class selection criteria; and responsive to determining that the class selection metrics satisfy the class selection criteria, provides the qubit constraint information to a quantum component of the system, receives quantumly measured values corresponding to expectation values of quantum operators acting on quantum states of qubits of the quantum component and representative of the expectation values of quantum operators acting on eigenstates of the active-space electronic Hamiltonian; and utilizes the measured values to approximate expectation values of quantum operators acting on eigenstates of the total electronic Hamiltonian to generate a model of the chemical system that represents a structural and/or chemical interaction characteristic of the chemical system.

### CROSS-REFERENCE TO RELATED APPLICATIONS

This application is a continuation in part of U.S. application Ser. No. 17/931,616, filed Sep. 13, 2022 and claims priority to U.S. Application No. 63/344,592, filed May 20, 2022, the content of which is hereby incorporated herein by reference in its entirety.

### TECHNICAL FIELD

The present disclosure relates to simulating chemical systems. An example embodiment relates to the use of hybrid quantum-classical computing for simulating chemical systems.

### BACKGROUND

A given chemical system, including, but not limited to, an atom, a molecule, an ion, a periodic solid, a periodic surface slab or a combination of these, consists of one or more atomic nuclei and at least one electron. The quantum state of said electrons is represented by an electronic wavefunction, which is conveniently expressed as a linear combination of Slater determinants, built from molecular (or crystal) orbitals.

As the number of electrons in an atom and/or atoms in a molecule, periodic solid, and/or periodic surface slab increases, the computational complexity and processing costs of modeling such systems increases significantly. Through applied effort, ingenuity, and innovation many deficiencies of modeling and/or simulating such systems have been solved by developing solutions that are structured in accordance with the embodiments of the present invention, many examples of which are described in detail herein.

### BRIEF SUMMARY OF EXAMPLE EMBODIMENTS

Various embodiments provide methods, systems, apparatus, computer program products, and/or the like for simulating chemical systems. Example embodiments provide methods, systems, apparatus, computer program products, and/or the like for simulating chemical systems that are capable of simulating chemical systems such as atoms, molecules, periodic solids, periodic surface slabs, and/or combinations thereof, more accurately than conventional methods by simulating their series of electronic states more comprehensively and accurately without creating in intractable computational task for computing hardware.

In various embodiments, a hybrid quantum-classical computing technique is used to accurately and efficiently model and/or simulate chemical systems. For example, in various embodiments, a first level simulation of the chemical system is used to identify and/or determine approximations to the electronic states of the chemical system, for example by means of the Hartree-Fock theory where the electronic wavefunction is defined by a single Slater determinant, yielding, inter alia, molecular orbitals. In various systems, a more accurate approximation may be constructed where said orbitals can be rotated and/or split into active and inactive orbitals, and the electronic wavefunction is defined by a linear combination of Slater determinants constructed by different occupations of said active orbitals. The inactive orbitals include the core and virtual orbitals.

Fermionic constraint information regarding the active orbitals is transformed into a qubit basis corresponding to the qubits of the quantum computing component. For example, in various embodiments, the fermionic constraint information comprises the active-space electronic Hamiltonian expressed in second quantization, i.e. via occupations of the active subset of orbitals. Using a quantum computing component, the information regarding the active-space Hamiltonian in the qubit basis is processed to determine a qubit representation of characteristics of the eigenstates of the active-space electronic Hamiltonian, in an example embodiment.

The qubit representation of the characteristics of the eigenstates of the active-space Hamiltonian are then translated into a classical representation of the characteristics of the eigenstates of the active-space Hamiltonian. For example, measurement operations are performed on a plurality of qubits of the quantum computing component and measured values corresponding to the expectation values of quantum operators acting on at least a portion of the plurality of qubits are determined therefrom. The classical representation of the characteristics of the eigenstates of the active-space Hamiltonian are then utilized to determine classically an approximation to the characteristics of the eigenstates of the total electronic Hamiltonian of the system, such as expectation values of quantum operators acting on such states, to complete the simulation of the chemical system, determine how the chemical system behaves in one or more interactions (e.g., with other chemical systems and/or with electromagnetic radiation), determine a structural characteristic of the chemical system, and/or the like.

In various embodiments, a chemical system is evaluated to determine whether the chemical system is a good candidate for generating a model of the chemical system using a hybrid quantum-classical approximation. For example, class selection metrics are evaluated by the classical computing component and it is determined whether the class selection metrics satisfy one or more class selection criteria, in an example embodiment. In various embodiments, when the class selection metrics do not satisfy the class selection criteria, a notification is provided and/or a model of the chemical system is generated using a classical approximation. In various embodiments, when the class selection metrics do satisfy the class selection criteria, a model of the chemical system is generated using a hybrid approximation. In various embodiments, the hybrid approximation used is selected based on the evaluation of type selection metrics and type selection criteria.

According to a first aspect, a method for simulating a chemical system using a hybrid quantum-classical computing system is provided. The method comprises determining, by a classical computing component of a hybrid quantum-classical computing system, fermionic constraint information regarding an active-space electronic Hamiltonian defined in the space of two or more active orbitals; translating, by the classical computing component, the fermionic constraint information regarding the active-space electronic Hamiltonian into a qubit basis to generate qubit constraint information regarding the active-space electronic Hamiltonian; providing, by the classical computing component, the qubit constraint information regarding active-space electronic Hamiltonian to a quantum computing component of the hybrid quantum-classical computing system; receiving, by the classical computing component, measured values corresponding to expectation values of quantum operators acting on quantum states of at least a portion of a plurality of qubits of the quantum computing component and representative of the expectation values of quantum operators acting on eigenstates of the active-space electronic Hamiltonian; and utilizing, by the classical computing component, the measured values representative of the expectation values of the quantum operators acting on the eigenstates of the active-space electronic Hamiltonian to yield an approximation to expectation values of quantum operators acting on eigenstates of the total electronic Hamiltonian to generate a model of the chemical system that represents at least one of: a structural characteristic of the chemical system, a chemical interaction characteristic of the chemical system, or a response characteristic of the chemical system.

In an example embodiment, the fermionic constraint information regarding the active-space electronic Hamiltonian defined in the space of two or more active orbitals comprises an effective fermionic Hamiltonian and the qubit constraint information regarding the active-space electronic Hamiltonian defined in the space of two or more active orbitals comprises a translated version of the fermionic Hamiltonian into the qubit basis.

In an example embodiment, the measured values comprise at least one of an expectation value of the active-space Hamiltonian or at least one reduced density matrix (RDM).

In an example embodiment, the at least one RDM comprises at least one of a one particle RDM (1-RDM), a two particle RDM (2-RDM), a three particle RDM (3-RDM), or a four particle RDM (4-RDM).

In an example embodiment, the method further comprises determining, by the classical computing component, an estimate of the 4-RDM based at least in part on at least one of the 1-RDM, 2-RDM, or 3-RDM.

In an example embodiment, utilizing the measured values representative of the expectation values of the quantum operators acting on the eigenstates of the active-space electronic Hamiltonian to yield an approximation to expectation values of quantum operators acting on eigenstates of the total electronic Hamiltonian comprises performing a second order N-electron Valence State Perturbation Theory calculation.

In an example embodiment, the one or more inactive orbitals comprise core orbitals and virtual orbitals.

In an example embodiment, the method further comprises performing, by the quantum computing component, state preparation of a plurality of qubits based at least in part on the qubit constraint information regarding the active-space Hamiltonian defined in the space of two or more active orbitals; and performing, by the quantum computing component, one or more measurement operations to determine the measured values based on quantum states of at least a portion of the plurality of qubits.

In an example embodiment, the method further comprises identifying, by the classical computing component, a plurality of orbitals of the chemical system; and partitioning the plurality of orbitals into the two or more active orbitals and the one or more inactive orbitals.

In an example embodiment, the quantum computing component is configured to use up to one hundred qubits to perform a quantum circuit.

In an example embodiment, the method further comprises causing, by the classical computing component, at least one of (a) display of a graphical representation of at least a portion of the model of the chemical system or (b) generation and storage in a classical memory of a file comprising one or more parameters of the model of the chemical system.

In an example embodiment, the one or more parameters of the model of the chemical system include the at least one of a structural characteristic of the chemical system, a chemical interaction characteristic of the chemical system, or a response characteristic of the chemical system.

According to another aspect, a hybrid quantum-classical computing system configured for simulating a chemical system is provided. In an example embodiment, the hybrid quantum-classical computing system comprises a classical computing component and a quantum computing component. The hybrid quantum-classical computing component is configured to use the classical computing component to determine fermionic constraint information regarding the active-space electronic Hamiltonian defined in the space of two or more active orbitals of the chemical system; translate the fermionic constraint information regarding the active-space electronic Hamiltonian defined in the space of two or more active orbitals into a qubit basis to generate qubit constraint information regarding the active-space electronic Hamiltonian defined in the space of two or more active orbitals; provide the qubit constraint information regarding the active-space electronic Hamiltonian to a quantum computing component of the hybrid quantum-classical computing system; receive measured values corresponding to the expectation values of quantum operators acting on quantum states of at least a portion of a plurality of qubits of the quantum computing component and representative of the expectation values of quantum operators acting on eigenstates of the active-space electronic Hamiltonian; and utilize the measured values representative of the expectation values of the quantum operators acting on the eigenstates of the active-space electronic Hamiltonian to yield an approximation to expectation values of quantum operators acting on eigenstates of the total electronic Hamiltonian to generate a model of the chemical system that represents at least one of: a structural characteristic of the chemical system, a chemical interaction characteristic of the chemical system, or a response characteristic of the chemical system.

In an example embodiment, the fermionic constraint information regarding the active-space electronic Hamiltonian defined in the space of two or more active orbitals comprises an effective fermionic Hamiltonian and the qubit constraint information regarding the active-space electronic Hamiltonian defined in the space of two or more active orbitals comprises a translated version of the effective fermionic Hamiltonian into the qubit basis.

In an example embodiment, the measured values comprise at least one of an expectation value of the active-space Hamiltonian or at least one reduced density matrix (RDM).

In an example embodiment, the at least one RDM comprises at least one of a one particle RDM (1-RDM), a two particle RDM (2-RDM), a three particle RDM (3-RDM), or a four particle RDM (4-RDM).

In an example embodiment, the hybrid quantum-classical computing system is further configured to use the classical computing component to determine an estimate of the 4-RDM based at least in part on at least one of the 1-RDM, 2-RDM, or 3-RDM.

In an example embodiment, utilizing the measured values representative of the expectation values of the quantum operators acting on the eigenstates of the active-space electronic Hamiltonian to yield an approximation to expectation values of quantum operators acting on eigenstates of the total electronic Hamiltonian comprises performing a second order N-electron Valence State Perturbation Theory calculation or an AC0 calculation.

In an example embodiment, the one or more inactive orbitals comprise core orbitals and virtual orbitals.

In an example embodiment, the hybrid quantum-classical computing system is further configured to use the quantum computing component to perform state preparation of a plurality of qubits based at least in part on the qubit constraint information regarding the active-space Hamiltonian defined in the space of two or more active orbitals; and perform one or more measurement operations to determine the measured values based on quantum states of at least a portion of the plurality of qubits.

In an example embodiment, the hybrid quantum-classical computing system is further configured to use the classical computing component to identify and optionally rotate a plurality of orbitals of the chemical system; and partition the plurality of orbitals into the two or more active orbitals and the one or more inactive orbitals.

In an example embodiment, the quantum computing component is configured to use up to one hundred qubits to perform a quantum circuit.

In an example embodiment, the hybrid quantum-classical computing system is further configured to use the classical computing component to cause at least one of (a) display of a graphical representation of at least a portion of the model of the chemical system or (b) generation and storage in a classical memory of a file comprising one or more parameters of the model of the chemical system.

In an example embodiment, the one or more parameters of the model of the chemical system include the at least one of a structural characteristic of the chemical system, a chemical interaction characteristic of the chemical system, or a response characteristic of the chemical system.

According to another aspect, a computer program product comprising at least one non-transitory computer-readable medium storing computer-readable instructions is provided. The computer-readable instructions are configured, when executed by a hybrid quantum-classical computing system comprising a classical computing component and a quantum computing component, cause the hybrid quantum-classical computing system to use the classical computing component to determine fermionic constraint information regarding the active-space electronic Hamiltonian defined in the space of two or more active orbitals of the chemical system; translate the fermionic constraint information regarding the active-space electronic Hamiltonian defined in the space of two or more active orbitals into a qubit basis to generate qubit constraint information regarding the active-space electronic Hamiltonian defined in the space of two or more active orbitals; provide the qubit constraint information regarding the active-space electronic Hamiltonian to a quantum computing component of the hybrid quantum-classical computing system; receive measured values corresponding to the expectation values of quantum operators acting on quantum states of at least a portion of a plurality of qubits of the quantum computing component and representative of the expectation values of quantum operators acting on eigenstates of the active-space electronic Hamiltonian; and utilize the measured values representative of the expectation values of the quantum operators acting on the eigenstates of the active-space electronic Hamiltonian to yield an approximation to expectation values of quantum operators acting on eigenstates of the total electronic Hamiltonian to generate a model of the chemical system that represents at least one of: a structural characteristic of the chemical system, a chemical interaction characteristic of the chemical system, or a response characteristic of the chemical system.

In an example embodiment, the fermionic constraint information regarding the active-space electronic Hamiltonian defined in the space of two or more active orbitals comprises an effective fermionic Hamiltonian and the qubit constraint information regarding the active-space electronic Hamiltonian defined in the space of two or more active orbitals comprises a translated version of the fermionic Hamiltonian into the qubit basis.

In an example embodiment, the measured values comprise at least one of an expectation value of the active-space Hamiltonian or at least one reduced density matrix (RDM).

In an example embodiment, the at least one RDM comprises at least one of a one particle RDM (1-RDM), a two particle RDM (2-RDM), a three particle RDM (3-RDM), or a four particle RDM (4-RDM).

In an example embodiment, the computer-readable instructions are further configured, when executed by the hybrid quantum-classical computing system, to cause the hybrid quantum-classical computing system to use the classical computing component to determine an estimate of the 4-RDM based at least in part on at least one of the 1-RDM, 2-RDM, or 3-RDM.

In an example embodiment, utilizing the measured values representative of the expectation values of the quantum operators acting on the eigenstates of the active-space electronic Hamiltonian to yield an approximation to expectation values of quantum operators acting on eigenstates of the total electronic Hamiltonian comprises performing a second order N-electron Valence State Perturbation Theory calculation.

In an example embodiment, the one or more inactive orbitals comprise core orbitals and virtual orbitals.

In an example embodiment, the computer-readable instructions are further configured, when executed by the hybrid quantum-classical computing system, to cause the hybrid quantum-classical computing system to use the quantum computing component to perform state preparation of a plurality of qubits based at least in part on the qubit constraint information regarding the active-space Hamiltonian defined in the space of two or more active orbitals; and perform one or more measurement operations to determine the measured values based on quantum states of at least a portion of the plurality of qubits.

In an example embodiment, the computer-readable instructions are further configured, when executed by the hybrid quantum-classical computing system, to cause the hybrid quantum-classical computing system to use the classical computing component to identify a plurality of orbitals of the chemical system; and partition the plurality of orbitals into the two or more active orbitals and the one or more inactive orbitals.

In an example embodiment, the quantum computing component is configured to use up to one hundred qubits to perform a quantum circuit.

In an example embodiment, the computer-readable instructions are further configured, when executed by the hybrid quantum-classical computing system, to cause the hybrid quantum-classical computing system to use the classical computing component to cause at least one of (a) display of a graphical representation of at least a portion of the model of the chemical system or (b) generation and storage in a classical memory of a file comprising one or more parameters of the model of the chemical system.

In an example embodiment, the one or more parameters of the model of the chemical system include the at least one of a structural characteristic of the chemical system, a chemical interaction characteristic of the chemical system, or a response characteristic of the chemical system.

According to another aspect a hybrid quantum-classical computing system is provided. The hybrid quantum-classical computing system comprises a classical computing component configured to determine at least an inactive portion of a model of a chemical system, wherein the inactive portion of the model of the chemical system represents one or more inactive orbitals of the chemical system. The hybrid quantum-classical computing system further comprises a quantum computing component configured to determine at least an active portion of the model of the chemical system, wherein the active portion of the model of the chemical system represents attributes of active orbitals of the chemical system determined based on translating fermionic constraint information regarding the active-space electronic Hamiltonian defined in a space of two or more active orbitals into a qubit basis to provide qubit constraint information corresponding to the active-space electronic Hamiltonian in a space of a plurality of qubits of the quantum computing component and performing a quantum circuit using the plurality of qubits and based at least in part on the qubit constraint information.

In an example embodiment, the fermionic constraint information regarding the active-space electronic Hamiltonian defined in the space of two or more active orbitals uses a spinless representation of the two or more active orbitals.

In an example embodiment, the quantum computing component is configured to use an Ansatz that is a symmetry-adapted singlet unitary coupled-cluster singles and doubles (UCCSD) ansatz.

In an example embodiment, the classical computing component is configured to use a cumulant expansion to generate at least one approximation of at least one multi-particle RDM.

In an example embodiment, the classical computing component is configured to generate a four particle RDM (4-RDM) from at least one of a one particle RDM (1-RDM), two particle RDM (2-RDM), or three particle RDM (3-RDM) measured by the quantum computing component.

In an example embodiment, the classical computing component is configured to compute a NEVPT2 or AC0 energy based at least in part on measurements indicating RDM values, the measurements captured as part of performing the quantum circuit.

In an example embodiment, the hybrid quantum-classical computing system is configured to define an active space corresponding to at least two active orbitals and a basis set corresponding to the chemical system; construct an active-space electronic Hamiltonian defined in the active space; define a corresponding ansatz representative of the two or more active orbitals of the chemical system, and determine parameters of the chemical system by using a VQE method applied to a quantum circuit generated from the active-space electronic Hamiltonian and provided with the Ansatz as initial quantum computation parameters. In various embodiments, the basis set defined is the functional format used to represent the orbitals (e.g., atomic Gaussian-type orbitals, Wannier functions, plane-waves, and/or the like).

According to another aspect a method performed by a hybrid quantum-classical computing system to generate a model of a chemical system is provided. The hybrid quantum-classical computing system comprises a classical computing component coupled to a quantum computing component. In an example embodiment, the method comprises representing a total electronic Hamiltonian and an active-space electronic Hamiltonian of the chemical system in the classical computing component; and representing active space wavefunctions of the chemical system and at least one active space Reduced Density Matrix (RDM) of the chemical system in the quantum computing component based at least in part on a translation of the active-space electronic Hamiltonian into a qubit basis of a plurality of qubits of the quantum computing component. The classical computing component uses the at least one active space RDM to determine an approximation of at least one additional RDM that represents at least one of a structural characteristic of the chemical system, a chemical interaction characteristic of the chemical system, or a response characteristic of the chemical system.

In an example embodiment, the method includes configuring the hybrid quantum-classical computing system to use a spinless formulation of the at least one active space RDM and the at least one additional RDM when computing characteristics of the chemical system by using the quantum computing component.

In an example embodiment, the method includes configuring the quantum computing component to use an Ansatz that is a symmetry-adapted singlet UCCSD.

In an example embodiment, the method includes configuring the hybrid quantum-classical computing system to use cumulant expansion to generate the approximation of the at least one additional RDM.

In an example embodiment, the method includes configuring the hybrid quantum-classical computing system to generate a 4-RDM from 1,2,3-RDM's.

In an example embodiment, the method includes configuring the hybrid quantum-classical computing system to compute a NEVPT2 or AC0 energy using quantumly-measured RDM's.

In an example embodiment, the method includes configuring the hybrid quantum-classical computing system to define an active space corresponding to at least two active orbitals and a basis set corresponding to the chemical system; construct an active-space electronic Hamiltonian defined in the active space; define a corresponding ansatz representative of the two or more active orbitals of the chemical system, and determine parameters of the chemical system by using a VQE method applied to a quantum circuit generated from the active-space electronic Hamiltonian and provided with the Ansatz as initial quantum computation parameters.

According to another aspect, a machine-readable data storage medium comprising specific instructions that are executable on data processing hardware is provided. The instructions, when executed by the data processing hardware, cause a classical computing component of a hybrid quantum-classical computing system to represent a total electronic Hamiltonian and an active space Hamiltonian of the chemical system in the classical computing component; and represent active space wavefunctions of the chemical system and at least one active space Reduced Density Matrix (RDM) of the chemical system in the quantum computing component based at least in part on a translation of the active-space electronic Hamiltonian into a qubit basis of a plurality of qubits of the quantum computing component. The classical computing component uses the at least one active space RDM to determine an approximation of at least one additional RDM that represents at least one of a structural characteristic of the chemical system, a chemical interaction characteristic of the chemical system, or a response characteristic of the chemical system.

According to another aspect, a method for simulating a chemical system using a hybrid quantum-classical computing system is provided. In an example embodiment, the method comprises obtaining, by a classical computing component of the hybrid quantum-classical computing system, an indication of a chemical system; evaluating, by the classical computing component, class selection metrics based at least in part on a structure of the chemical system; determining, by the classical computing component, whether the class selection metrics satisfy one or more class selection criteria; and responsive to determining that class selection metrics do not satisfy the one or more class selection criteria, performing, by the classical computing component, at least one of (a) providing a notification that the class selection criteria are not satisfied by the chemical system or (b) performing a classical approximation to generate a model of the chemical system that represents at least one of: a structural characteristic of the chemical system, a chemical interaction characteristic of the chemical system, or a response characteristic. The method further includes, responsive to determining that the class selection metrics do satisfy the one or more class selection criteria providing, by the classical computing component, qubit constraint information regarding an active-space electronic Hamiltonian for the chemical system to a quantum computing component of the hybrid quantum-classical computing system; receiving, by the classical computing component, measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and utilizing, by the classical computing component, the measured values to generate the model of the chemical system that represents at least one of: a structural characteristic of the chemical system, a chemical interaction characteristic of the chemical system, or a response characteristic.

In an example embodiment, evaluating the class selection metrics comprises performing a multireference diagnostic of the chemical system to determine a multireference diagnostic number corresponding to the chemical system and determining whether the class selection metrics satisfy the one or more class selection criteria comprises determining whether the multireference diagnostic number corresponding to the chemical system is greater than a threshold strength.

In an example embodiment, performing the multireference diagnostic of the chemical system comprises determining a classical approximation of a multireference wave function of the chemical system and determining a degree of electron correlation of the chemical system based at least in part on the classical approximation of the multireference wave function.

In an example embodiment, the method further comprises causing, by the classical computing component, at least one of (a) display of a graphical representation of at least a portion of the model of the chemical system or (b) generation and storage in a classical memory of a file comprising one or more parameters of the model of the chemical system.

In an example embodiment, the method further comprises, responsive to determining that the one or more class selection criteria are satisfied, evaluating, by the classical computing entity, type selection metrics based on one or more of (a) at least a portion of a structure of the chemical system, (b) information about a quantum computing component of the hybrid quantum-classical computing system, or (c) user input; and selecting a hybrid approximation based on one or more type selection criteria and the type selection metrics, wherein the classical computing component uses the hybrid approximation to generate the model of the chemical system utilizing the measured values.

In an example embodiment, the second selection criteria are evaluated at least in part based on a number of active electrons of the chemical system, a number of active orbitals of the chemical system, or one or more symmetries of the chemical system.

In an example embodiment, the second selection criteria are evaluated at least in part based on a number of qubits of the quantum computing component, available run time of the quantum computing component, or a noise profile of one or more functions of the quantum computing component.

In an example embodiment, the hybrid approximation is one of a second-order N-electron valence state perturbation theory (NEVPT2) approximation or an AC0 approximation.

In an example embodiment, the measured values that are measured by the quantum computing component are determined based on the hybrid approximation.

In an example embodiment, the measured values comprise at least one of an expectation value of the active-space Hamiltonian or at least one reduced density matrix (RDM).

In an example embodiment, the at least one RDM comprises at least one of a one particle RDM (1-RDM), a two particle RDM (2-RDM), a three particle RDM (3-RDM), or a four particle RDM (4-RDM).

In an example embodiment, the method further comprises generating the qubit constraint information, wherein generating the qubit constrain information comprises determining, by the classical computing component, fermionic constraint information regarding an active-space electronic Hamiltonian defined in an active space of two or more active orbitals of the chemical system; and translating, by the classical computing component, the fermionic constraint information regarding the active-space electronic Hamiltonian into a qubit basis to generate qubit constraint information regarding the active-space electronic Hamiltonian.

In an example embodiment, the fermionic constraint information regarding the active-space electronic Hamiltonian defined in the active space of two or more active orbitals of the chemical system is performed as part of evaluating the class selection metrics.

In an example embodiment, the method further comprises performing, by the quantum computing component, state preparation of a plurality of qubits based at least in part on the qubit constraint information regarding the active-space electronic Hamiltonian; and performing, by the quantum computing component, one or more measurement operations to determine the measured values based on quantum states of at least a portion of the plurality of qubits.

According to another aspect, a hybrid quantum-classical computing system configured for simulating a chemical system is provided. In an example embodiment, the hybrid quantum-classical computing system comprises a classical computing component and a quantum computing component. The hybrid quantum-classical computing system is configured to use the classical computing component to obtain an indication of a chemical system; evaluate class selection metrics based at least in part on a structure of the chemical system; determine whether the class selection metrics satisfy one or more class selection criteria; and responsive to determining that class selection metrics do not satisfy the one or more class selection criteria, perform at least one of (a) providing a notification that the class selection criteria are not satisfied by the chemical system or (b) performing a classical approximation to generate a model of the chemical system that represents at least one of: a structural characteristic of the chemical system, a chemical interaction characteristic of the chemical system, or a response characteristic. The hybrid quantum-classical computing system is further configured to use the classical computing component to, responsive to determining that the class selection metrics do satisfy the one or more class selection criteria provide qubit constraint information regarding an active-space electronic Hamiltonian for the chemical system to a quantum computing component of the hybrid quantum-classical computing system; receive measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and utilize the measured values to generate the model of the chemical system that represents at least one of a structural characteristic of the chemical system, a chemical interaction characteristic of the chemical system, or a response characteristic.

In an example embodiment, evaluating the class selection metrics comprises performing a multireference diagnostic of the chemical system to determine a multireference diagnostic number corresponding to the chemical system and determining whether the class selection metrics satisfy the one or more class selection criteria comprises determining whether the multireference diagnostic number corresponding to the chemical system is greater than a threshold strength.

In an example embodiment, performing the multireference diagnostic of the chemical system comprises determining a classical approximation of a multireference wave function of the chemical system and determining a degree of electron correlation of the chemical system based at least in part on the classical approximation of the multireference wave function.

In an example embodiment, the hybrid quantum-classical computing system is further configured to use the classical computing component to cause at least one of (a) display of a graphical representation of at least a portion of the model of the chemical system or (b) generation and storage in a classical memory of a file comprising one or more parameters of the model of the chemical system.

In an example embodiment, the hybrid quantum-classical computing system is further configured to use the classical computing component to, responsive to determining that the one or more class selection criteria are satisfied, evaluate type selection metrics based on one or more of (a) at least a portion of a structure of the chemical system, (b) information about a quantum computing component of the hybrid quantum-classical computing system, or (c) user input; and selecting a hybrid approximation based on one or more type selection criteria and the type selection metrics, wherein the classical computing component uses the hybrid approximation to generate the model of the chemical system utilizing the measured values.

In an example embodiment, the second selection criteria are evaluated at least in part based on a number of active electrons of the chemical system, a number of active orbitals of the chemical system, or one or more symmetries of the chemical system.

In an example embodiment, the second selection criteria are evaluated at least in part based on a number of qubits of the quantum computing component, available run time of the quantum computing component, or a noise profile of one or more functions of the quantum computing component.

In an example embodiment, the hybrid approximation is one of a second-order N-electron valence state perturbation theory (NEVPT2) approximation or an AC0 approximation.

In an example embodiment, the measured values that are measured by the quantum computing component are determined based on the hybrid approximation.

In an example embodiment, the measured values comprise at least one of an expectation value of the active-space Hamiltonian or at least one reduced density matrix (RDM).

In an example embodiment, the at least one RDM comprises at least one of a one particle RDM (1-RDM), a two particle RDM (2-RDM), a three particle RDM (3-RDM), or a four particle RDM (4-RDM).

In an example embodiment, the hybrid quantum-classical computing system is further configured to use the classical computing component to generate the qubit constraint information, wherein generating the qubit constrain information comprises determining fermionic constraint information regarding an active-space electronic Hamiltonian defined in an active space of two or more active orbitals of the chemical system; and translating the fermionic constraint information regarding the active-space electronic Hamiltonian into a qubit basis to generate qubit constraint information regarding the active-space electronic Hamiltonian.

In an example embodiment, the fermionic constraint information regarding the active-space electronic Hamiltonian defined in the active space of two or more active orbitals of the chemical system is performed as part of evaluating the class selection metrics.

In an example embodiment, the hybrid quantum-classical computing system is further configured to use the classical computing component to perform state preparation of a plurality of qubits based at least in part on the qubit constraint information regarding the active-space electronic Hamiltonian; and performing, by the quantum computing component, one or more measurement operations to determine the measured values based on quantum states of at least a portion of the plurality of qubits.

According to another aspect, a machine-readable data storage medium comprising specific instructions that are executable on data processing hardware is provided. The instructions, when executed by the data processing hardware, cause a classical computing component of a hybrid quantum-classical computing system to obtain an indication of a chemical system; evaluate class selection metrics based at least in part on a structure of the chemical system; determine whether the class selection metrics satisfy one or more class selection criteria; and responsive to determining that class selection metrics do not satisfy the one or more class selection criteria, perform at least one of (a) providing a notification that the class selection criteria are not satisfied by the chemical system or (b) performing a classical approximation to generate a model of the chemical system that represents at least one of: a structural characteristic of the chemical system, a chemical interaction characteristic of the chemical system, or a response characteristic. The instructions, when executed by the data processing hardware, further cause the classical computing component to, responsive to determining that the class selection metrics do satisfy the one or more class selection criteria provide qubit constraint information regarding an active-space electronic Hamiltonian for the chemical system to a quantum computing component of the hybrid quantum-classical computing system; receive measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and utilize the measured values to generate the model of the chemical system that represents at least one of a structural characteristic of the chemical system, a chemical interaction characteristic of the chemical system, or a response characteristic.

According to still another aspect, a method for simulating a chemical system using a hybrid quantum-classical computing system is provided. In an example embodiment, the method includes obtaining, by a classical computing component of the hybrid quantum-classical computing system, an indication of a chemical system; evaluating, by the classical computing component, selection metrics based on one or more of (a) at least a portion of an atomic structure of the chemical system, (b) information about a quantum computing component of the hybrid quantum-classical computing system, or (c) user input; selecting a hybrid approximation based on one or more type selection criteria and the selection metrics; providing, by the classical computing component, qubit constraint information regarding an active-space electronic Hamiltonian for the chemical system to a quantum computing component of the hybrid quantum-classical computing system; receiving, by the classical computing component, measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and utilizing, by the classical computing component, the measured values and the hybrid approximation to generate a model of the chemical system that represents at least one of: a structural characteristic of the chemical system, a chemical interaction characteristic of the chemical system, or a response characteristic.

In an example embodiment, the second selection criteria are evaluated at least in part based on a number of active electrons of the chemical system, a number of active orbitals of the chemical system, or one or more symmetries of the chemical system.

In an example embodiment, the second selection criteria are evaluated at least in part based on a number of qubits of the quantum computing component, available run time of the quantum computing component, or a noise profile of one or more functions of the quantum computing component.

In an example embodiment, the hybrid approximation is one of a second-order N-electron valence state perturbation theory (NEVPT2) approximation or an AC0 approximation.

In an example embodiment, the measured values that are measured by the quantum computing component are determined based on the hybrid approximation.

In an example embodiment, the measured values comprise at least one of an expectation value of the active-space Hamiltonian or at least one reduced density matrix (RDM).

According to another aspect, a hybrid quantum-classical computing system configured for simulating a chemical system is provided. In an example embodiment, the hybrid quantum-classical computing system comprises a classical computing component and a quantum computing component. The hybrid quantum-classical computing system is configured to use the classical computing component to obtain an indication of a chemical system; evaluate selection metrics based on one or more of (a) at least a portion of an atomic structure of the chemical system, (b) information about a quantum computing component of the hybrid quantum-classical computing system, or (c) user input; select a hybrid approximation based on one or more type selection criteria and the selection metrics; provide qubit constraint information regarding an active-space electronic Hamiltonian for the chemical system to a quantum computing component of the hybrid quantum-classical computing system; receive measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and utilize the measured values and the hybrid approximation to generate a model of the chemical system that represents at least one of: a structural characteristic of the chemical system, a chemical interaction characteristic of the chemical system, or a response characteristic.

In an example embodiment, the second selection criteria are evaluated at least in part based on a number of active electrons of the chemical system, a number of active orbitals of the chemical system, or one or more symmetries of the chemical system.

In an example embodiment, the second selection criteria are evaluated at least in part based on a number of qubits of the quantum computing component, available run time of the quantum computing component, or a noise profile of one or more functions of the quantum computing component.

In an example embodiment, the hybrid approximation is one of a second-order N-electron valence state perturbation theory (NEVPT2) approximation or an AC0 approximation.

In an example embodiment, the measured values that are measured by the quantum computing component are determined based on the hybrid approximation.

In an example embodiment, the measured values comprise at least one of an expectation value of the active-space Hamiltonian or at least one reduced density matrix (RDM).

According to another aspect, a machine-readable data storage medium comprising specific instructions that are executable on data processing hardware is provided. The instructions, when executed by the data processing hardware, cause a classical computing component of a hybrid quantum-classical computing system to obtain an indication of a chemical system; evaluate selection metrics based on one or more of (a) at least a portion of an atomic structure of the chemical system, (b) information about a quantum computing component of the hybrid quantum-classical computing system, or (c) user input; select a hybrid approximation based on one or more type selection criteria and the selection metrics; provide qubit constraint information regarding an active-space electronic Hamiltonian for the chemical system to a quantum computing component of the hybrid quantum-classical computing system; receive measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and utilize the measured values and the hybrid approximation to generate a model of the chemical system that represents at least one of: a structural characteristic of the chemical system, a chemical interaction characteristic of the chemical system, or a response characteristic.

### BRIEF DESCRIPTION OF THE SEVERAL VIEWS OF THE DRAWING(S)

Having thus described the invention in general terms, reference will now be made to the accompanying drawings, which are not necessarily drawn to scale, and wherein:

FIG. 1 is a schematic diagram illustrating an example hybrid quantum-classical computing system, according to an example embodiment.

FIG. 2 is a flowchart illustrating processes, procedures, and/or operations performed by a classical component of a hybrid quantum-classical computing system to generate and/or provide a model of a chemical system, according to an example embodiment.

FIG. 3 is a flowchart illustrating processes, procedures, and/or operations performed by a classical component of a hybrid quantum-classical computing system to preform perform pre-processing to generate qubit constraint information, according to an example embodiment.

FIG. 4 is a flowchart illustrating processes, procedures, and/or operations performed by a controller of a quantum component of a hybrid quantum-classical computing system, according to an example embodiment.

FIG. 5 provides a schematic diagram of an example controller of a quantum component of a hybrid-classical computing system that is configured to control operation of one or more elements of the quantum component, according to various embodiments.

FIG. 6 provides a schematic diagram of an example classical component of a hybrid quantum-classical computing system that may be used in accordance with an example embodiment.

FIGS. 7A and 7B provide plots illustrating comparison of an example chemical system model generated via an example embodiment with conventional models of the example chemical system.

FIG. 8 provides plot illustrating comparison of two example chemical system models generated via example embodiments with a conventional model of the example chemical system.

### DETAILED DESCRIPTION OF SOME EXAMPLE EMBODIMENTS

The present invention now will be described more fully hereinafter with reference to the accompanying drawings, in which some, but not all embodiments of the invention are shown. Indeed, the invention may be embodied in many different forms and should not be construed as limited to the embodiments set forth herein; rather, these embodiments are provided so that this disclosure will satisfy applicable legal requirements. The term “or” (also denoted “I”) is used herein in both the alternative and conjunctive sense, unless otherwise indicated. The terms “illustrative” and “exemplary” are used to be examples with no indication of quality level. The terms “generally,” “substantially,” and “approximately” refer to within engineering and/or manufacturing tolerances and/or within user measurement capabilities, unless otherwise indicated. Like numbers refer to like elements throughout.

Various embodiments provide methods, systems, apparatus, computer program products, and/or the like for simulating chemical systems. Example embodiments provide methods, systems, apparatus, computer program products, and/or the like for simulating chemical systems that is capable of simulating atoms, molecules, solids, periodic atomic systems, and/or the like more accurately than conventional methods by simulating their orbitals, and in particular their active orbitals, more comprehensively and accurately without creating an intractable computational task for computing hardware. For example, various embodiments, provide methods, systems, apparatus, computer program products, and/or the like for generating models of chemical systems that represent at least one of a structural characteristic of the chemical system, a chemical interaction characteristic of the chemical system, or a response characteristic of the chemical system.

In various embodiments, a hybrid quantum-classical computing technique is used to accurately and efficiently model and/or simulate chemical systems. For example, in various embodiments, a first level simulation of the chemical system is used to identify and/or determine approximations to the electronic states of the chemical system, for example by means of the Hartree-Fock theory where the electronic wavefunction is defined by a single Slater determinant, yielding, inter alia, molecular orbitals. In various systems, a more accurate approximation may be constructed where said orbitals can be rotated and split into active and inactive orbitals, and the electronic wavefunction is defined by a linear combination of Slater determinants constructed by different occupations of said active orbitals. The inactive orbitals include the core and virtual orbitals.

In various systems, the orbitals can be split into active and inactive orbitals based on the structural and/or interaction characteristics of the chemicals system being explored, evaluated, modeled, and/or the like. For example, an active space comprising and/or consisting of two or more active orbitals is defined and an inactive space comprising and/or consisting of one or more inactive orbitals is defined. The one or more inactive orbitals includes the core orbitals and the virtual orbitals.

Fermionic constraint information regarding the active-space electronic Hamiltonian defined in the space of two or more active orbitals is determined and translated and/or transformed into a qubit basis corresponding to the qubits of a quantum computing component. In various embodiments, the qubit basis is determined and/or dependent on the type of qubit used by the quantum component of the hybrid quantum-classical computing system being used. For example, the quantum component may use photons, electrons, atomic nuclei, neutral atoms, ions, Josephson junctions, quantum dots, topological anyons, and/or other quantum particles and/or systems as qubits. The qubit constraint information determined by translating and/or transforming the fermionic constraint information into the qubit basis will be dependent on the qubit basis corresponding to the particular qubits used by the quantum component. For example, in various embodiments, the fermionic constraint information comprises the active-space electronic Hamiltonian in terms of operators in the Hilbert space of the chemical system and/or the active space of the chemical system as defined by the two or more active orbitals. The qubit constraint information comprises the active-space electronic Hamiltonian in terms of operators in the Hilbert space of the qubits of the quantum computing component.

Using a quantum computing component, the qubit constraint information is processed to determine and/or generate a qubit representation of characteristics of the eigenstates of the active-space electronic Hamiltonian, in an example embodiment. For example, a quantum circuit is determined and executed that performs state preparation of a plurality of qubits of the quantum component such that the quantum states of the plurality of qubits are prepared in accordance with the active-space electronic Hamiltonian to represent one or more eigenstates (e.g., active orbital wavefunctions) and/or expectation values of the active-space electronic Hamiltonian. The prepared states of the qubits provide a qubit representation of characteristics of the eigenstates of the active-space electronic Hamiltonian, in an example embodiment. Measurement operations are performed by the quantum computing component to extract the qubit representation of characteristics of the eigenstates of the active-space electronic Hamiltonian, in an example embodiment. For example, measurement operations are performed on a plurality of qubits of the quantum computing component and measured values corresponding to the expectation values of quantum operators acting on at least a portion of the plurality of qubits are determined therefrom.

The qubit representation of the characteristics of the eigenstates of the active-space Hamiltonian are then translated into a classical representation of the characteristics of the eigenstates of the active-space Hamiltonian. For example, measured values corresponding to the characteristics of the eigenstates of the active-space Hamiltonian are determined based on the results of the measurement operations performed on the qubits.

For example, the classical representation of the characteristics of the eigenstates of the active-space Hamiltonian are then utilized to determine classically an approximation to the characteristics of the eigenstates of the total electronic Hamiltonian of the system, such as expectation values of quantum operators acting on such states, to complete the simulation of the chemical system, determine how the chemical system behaves in one or more interactions (e.g., with other chemical systems and/or electromagnetic radiation), determine a structural characteristics of the chemical system, and/or the like.

As used herein, a classical component or classical computer is a computing entity that uses semiconductor-based computational techniques and hardware. A quantum component or quantum computing component uses the quantum states of quantum particles (referred to as qubits) to perform computations.

In various embodiments, prior to the generation of the model, a chemical system is evaluated to determine whether the chemical system is a good candidate for generating a model of the chemical system using a hybrid quantum-classical approximation. For example, class selection metrics are evaluated by the classical computing component and it is determined whether the class selection metrics satisfy one or more class selection criteria, in an example embodiment. For example, based on whether or not the class selection metrics satisfy the class selection criteria is used by the classical computing component to determine which class of approximation (e.g., a hybrid approximation or a classical approximation) to use in generating the model of the chemical system. In various embodiments, when the class selection metrics do not satisfy the class selection criteria, a notification is provided and/or a model of the chemical system is generated using a classical approximation. In various embodiments, when the class selection metrics do satisfy the class selection criteria, a model of the chemical system is generated using a hybrid approximation. For example, in various embodiments, when a multireference diagnostic number (such as, but not limited to, T₁ diagnostic, or D₁ diagnostic etc.) corresponding to the chemical system satisfies (e.g., is greater than or equal to) a threshold of the class selection criteria, it is determined that the class selection metrics satisfies the one or more class selection criteria and a hybrid approximation is used to generate a model of the chemical system. In another example, in various embodiments, when a multireference diagnostic number corresponding to the chemical system does not satisfy (e.g., is less than) a threshold of the class selection criteria, it is determined that the class selection metrics does not satisfy the one or more class selection criteria and a hybrid approximation is not used to generate a model of the chemical system.

In various embodiments, the type of hybrid approximation used is selected based on the evaluation of type selection metrics and type selection criteria. For example, the type selection metrics are determined based on one or more of at least a portion of an atomic structure of the chemical system, information about a quantum computing component of the hybrid quantum-classical computing system, user input, and/or the like, in various embodiments. For example, a hybrid quantum-classical computing system may be configured to generate models of chemical systems using two or more hybrid approximations. The classical computing component of the hybrid quantum-classical computing system may select which of the two or more hybrid approximations are used to generate the model of the chemical system based on comparing the type selection metrics to the type selection criteria. In an example embodiment, the two or more hybrid approximations include second-order N-electron valence state perturbation theory (NEVPT2) and/or adiabatic connection (AC) with the first-order expansion of the AC integrand at the coupling constant equal to 0 (AC0).

Conventional classical computer software products are known that can be executed on classical computing hardware, for example on classical non-quantum computing components, to simulate chemical modules and to determine their manners of interactions with other molecules. Such computer software products are configured to compute properties of electronic states of a given atom, molecule or solid where said electronic states are represented by a single electronic configuration (Slater determinant) as a first category of computation, and then compute properties of said electronic states where these states are represented by electronic configurations (Slater determinants) obtained by exciting (promoting) one or more electrons from occupied electron states to unoccupied electron states, as a second category of computation.

An objective technical problem that is encountered in practice is that computing resources required for implementing the second category of computations can be very challenging; in certain situations, the amount of computing resources required can become intractable. As a result, approximations are conventionally often used when performing the computations associated with the aforesaid second category. In various scenarios, these approximations are not sufficiently accurate to provide structural and/or interaction characteristics of the atom or molecule to account for and/or predict real world observations of the atom or molecule and/or interactions thereof.

Quantum computing components are expected to provide systems that can perform complex computations in shortened time frames and with reduced memory requirements. However, currently operational quantum computing components tend to include relatively low numbers of qubits (e.g., less than 100 qubits) and tend to be relatively noisy. As a result, a further technical problem exists in that currently operational quantum computing components do not provide sufficient computational resources for performing complete chemical system simulations or even merely simulations of the excited states of a chemical system.

Various embodiments provide technical solutions to these technical problems. For example, in various embodiments, (quantumly) measured values for Hamiltonians defined in the space of occupations of the active orbitals are determined using the quantum component of the hybrid quantum-classical computing system. The static (i.e. non-dynamic) correlation energy arising from entanglement of the active orbitals has a large effect on the structural characteristics, interaction characteristics, and/or response characteristics of interest for the chemical system and is more computationally demanding to determine than the dynamic correlation energy arising from excitations into inactive virtual orbitals. However, the minimal number of active orbitals required for a satisfactory accurate model of a chemical system tends to be in a range of 2 to 100 orbitals.

Therefore, by limiting the problem passed to the quantum component to the active orbitals, a more accurate determination of the structural and/or interaction characteristics of interest for the chemical system can be obtained and the computational power of the quantum component used efficiently and effectively. Moreover, by using the quantum component to generate a qubit representation of the characteristics of the eigenstates of the active-space electronic Hamiltonian, rather than merely using the quantum component to generate a qubit representation of the characteristics of the eigenstates of the total electronic Hamiltonian, the accuracy and efficiency of the model of the chemical system is further improved. Thus, various embodiments provide improvements to the technical field of chemical system simulation by providing computationally tractable and more accurate simulations of the structural characteristics, interaction characteristics of chemical systems, and/or response characteristics of the chemical system.

For example, in various embodiments, for a given chemical system, an active space comprising two or more active orbitals and a basis set for the chemical system are defined. A fermionic Hamiltonian for the active space is determined using the defined basis set. State preparation of qubits of a quantum computing component is then performed such that the quantum states of the qubits represent characteristics of the eigenstates of the active-space electronic Hamiltonian of the chemical system. Measurement operations are performed such that at least one particle, two particle, and three particle reduced density matrices (1-RDM, 2-RDM, and 3-RDM) are determined through quantum measurements and/or measurements of quantum states of the qubits of the quantum computing component. The four particle reduced density matrix (4-RDM) may be determined through quantum measurements and/or measurements of quantum states of the qubits of the quantum computing component or through a cumulant expansion, in various embodiments. The classical computing component uses the at least one active space RDM to determine an approximation of at least one additional RDM and/or an NEVPT2 or AC0 energy that represents at least one of a structural characteristic of the chemical system, a chemical interaction characteristic of the chemical system, or a response characteristic of the chemical system.

Additionally, in various embodiments, prior to using quantum computing resources, a check is performed to determine whether the chemical system is a good candidate for the determination of a hybrid quantum-classical approximation model. For example, class selection metrics are determined based on structural characteristics of the chemical system and compared to class selection criteria to determine whether the chemical system is a good candidate for the determination of a hybrid quantum-classical approximation model. For example, when the class selection metrics satisfy the class selection criteria, it is expected that the use of a quantum computing component in the determination of the model of the chemical system will provide a more accurate and/or computationally efficient result. When the class selection metrics do not satisfy the class selection criteria, it is expected that the use of a quantum computing component in the determination of the model of the chemical system is not likely to provide an advantage and therefor, quantum computing resources may not be used to determine the model of the chemical system. This provides the advantage of ensuring the (limited) quantum computing resources (e.g., the quantum computing component of the hybrid quantum-classical computing system) are used efficiently and effectively, resulting in improved performance of the hybrid quantum-classical computing system.

Moreover, in various embodiments, the hybrid quantum-classical computing system is configured to select a hybrid approximation to be used in generating a model of a chemical system. For example, the hybrid quantum-classical computing system is configured to use two or more hybrid approximations in generating a model of a chemical system. The classical computing entity may evaluate type selection metrics and compare the type selection metrics to approximation selection criteria to select a hybrid approximation to be used in generating the model of the chemical system. In various embodiments, the type selection metrics are determined based on one or more of at least a portion of an atomic structure of the chemical system, information about a quantum computing component of the hybrid quantum-classical computing system, user input, and/or the like. Thus, a hybrid approximation that efficiently and effectively uses the (limited) quantum computing resources (e.g., the quantum computing component of the hybrid quantum-classical computing system) can be selected for use. This functionality of various embodiments results in improved performance of the hybrid quantum-classical computing system and improved user experience.

### Example Hybrid Quantum-Classical Computing System

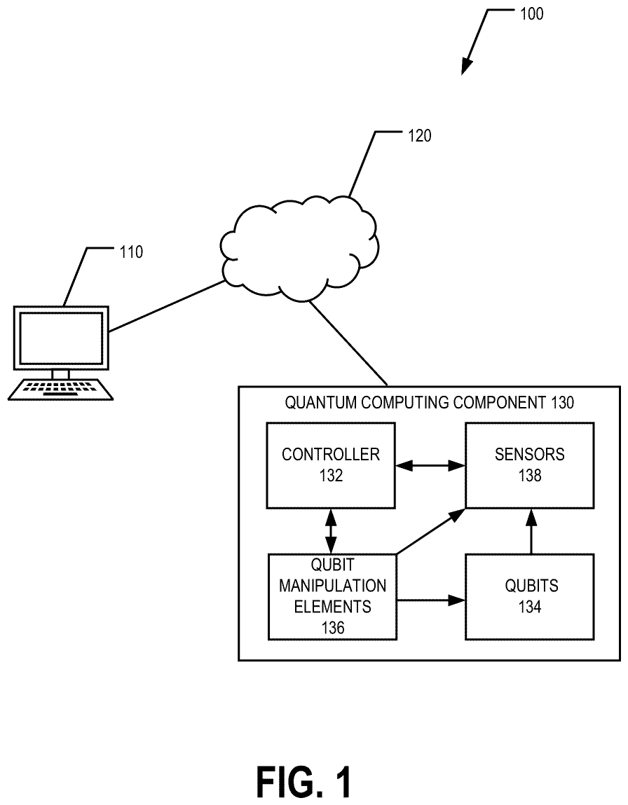

FIG. 1 provides a block diagram of an example hybrid quantum-classical computing system 100, in accordance with various embodiments. In various embodiments, the hybrid quantum-classical computing system 100 comprises a classical component such as a classical computing component 110 and a quantum component such as a quantum computing component 130.

The quantum computing component 130 comprises a controller 132, qubits 134, qubit manipulation elements 136 and sensors 138. The controller 132 is configured to control operation of the qubit manipulation elements 136 to cause desired manipulations (e.g., controlled quantum state evolution) of the qubits 134. The controller 132 is further configured to control operation of the sensors 138 that are configured to monitor, measure, and/or capture measurements corresponding to the operation of the qubit manipulation components and capture measurements indicating the respective quantum states of respective qubits 134.

For example, in various embodiments, the qubit manipulation elements 136 comprise voltage/current sources, laser sources, magnetic field sources (e.g., electromagnets and/or permanent magnets) and/or other hardware components configured for use in confining the qubits and/or manipulating the quantum state of the qubits. For example, in various embodiments, the sensors 138 comprise photodetectors, voltage/current sensors, temperature sensors, pressure sensors, and/or other sensors that may be used to determine a quantum state of a qubit and/or monitor operation of one or more of the qubit manipulation elements 136.

In various embodiments, the classical computing component 110 is in communication with the controller 132 of the quantum computing component 130 via one or more wired or wireless networks 120 and/or via direct wired and/or wireless communications. For example, the classical computing component 110 is configured to provide qubit constraint information to quantum computing component 130 (e.g., the controller 132 thereof) and receive measured values corresponding to expectation values of quantum operators acting on quantum states of at least a portion of a plurality of qubits of the quantum computing component and representative of the expectation values of quantum operators acting on eigenstates of the active-space electronic Hamiltonian that were provided by the quantum computing component 130 (e.g., the controller 132 thereof) via the one or more wired or wireless networks 120 and/or via direct wired and/or wireless communications between the classical computing component 110 and the quantum computing component 130. For example, the quantum computing component 130 is configured to receive qubit constraint information provided by the classical computing component 110 and provide measured values corresponding to expectation values of quantum operators acting on quantum states of at least a portion of a plurality of qubits of the quantum computing component and representative of the expectation values of quantum operators acting on eigenstates of the active-space electronic Hamiltonian for receipt by the classical computing component 110 via the one or more wired or wireless networks 120 and/or via direct wired and/or wireless communications between the classical computing component 110 and the quantum computing component 130.

### Example Operation of a Hybrid Quantum-Classical Computing System

In various embodiments, a hybrid quantum-classical computing system 100 is used to simulate a chemical system. For example, the hybrid quantum-classical computing system 100 is used to generate a model of a chemical system that represents one or more structural characteristics of the chemical system, one or more chemical interaction characteristics of the chemical system, one or more response characteristics of the chemical system, and/or the like, in various embodiments. In various embodiments, the model, a portion thereof, and/or a graphical representation thereof is displayed via a display (e.g., of the classical computing component 110), stored to a file that may be used as an input and/or provided directly as input to other simulations or models that use the interaction, structural characteristics of the chemical system, and/or response characteristics of the chemical system to perform one or more functions thereof, and/or the like.

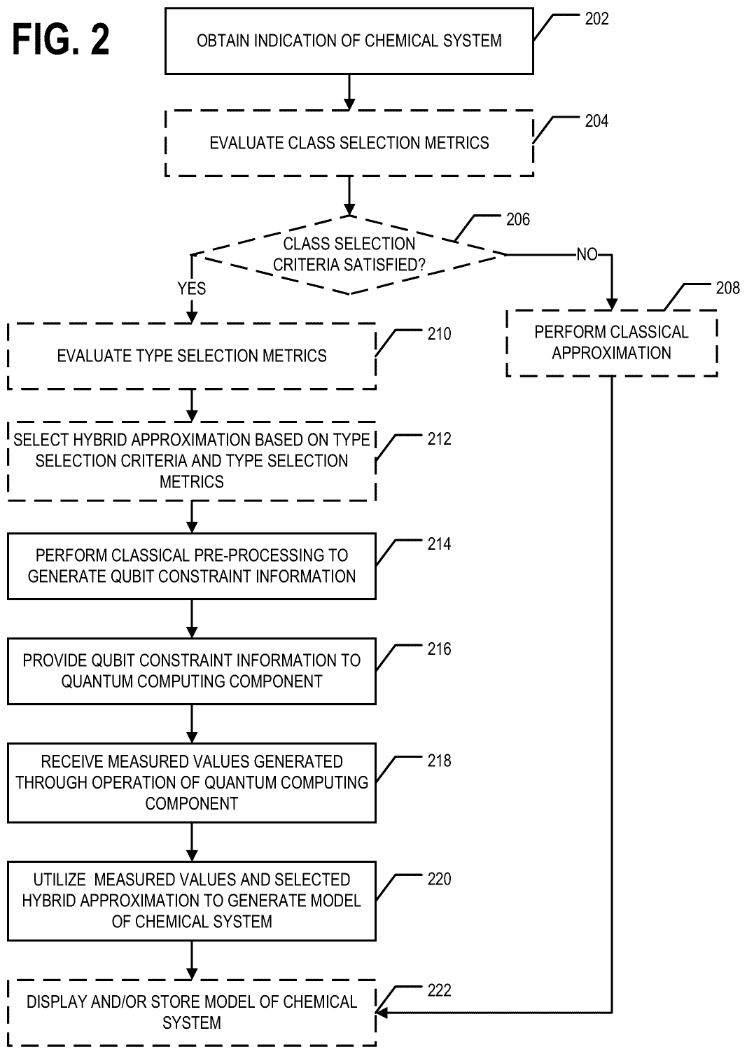

FIG. 2 provides a flowchart of various processes, procedures, operations, and/or the like performed by the classical computing component 110, for example, to generate and provide a model of a chemical system. Starting at step/operation 202, in various embodiments, the classical computing component 110 obtains information corresponding to, indicating, and/or identifying a chemical system. For example, user input (e.g., received via a user input interface of the classical computing component 110) may provide, select, and/or cause the accessing of information corresponding to a chemical system. In an example embodiment, the information corresponding to the chemical system may be a chemical formula for the chemical system (H, H₂O, NH⁴⁺, etc.) and/or other designation of the chemical system. In an example embodiment, the information corresponding to the chemical system includes nuclei information for each atomic nucleus of the chemical system (e.g., numbers of protons and neutrons present in the respective atomic nuclei), a number of electrons in the chemical system, Cartesian coordinates of the nuclei, and/or any other information used to define the chemical system.

In various embodiments, obtaining of the information corresponding to the chemical system triggers and/or causes the classical computing component 110 to determine and/or generate a model of the chemical system that represents one or more structural characteristics of the chemical system, one or more chemical interaction characteristics of the chemical system, one or more response characteristics of the chemical system, and/or the like. For example, the classical computing component 110 may determine and/or identify the active orbitals of the chemical system and provide information corresponding to the active orbitals to the quantum computing component 130. The quantum computing component 130 may use the information corresponding to the active orbitals to determine one or more measured values based on a qubit representation of characteristics of the active orbitals. For example, in various embodiments, the one or more measured values comprise at least one of an expectation value of the active-space Hamiltonian or at least one reduced density matrix (RDM). For example, in various embodiments, the one or more measured values are representative of the expectation values of the quantum operators acting on the eigenstates of the active-space electronic Hamiltonian.

Given the limited number of active orbitals (e.g., generally less than 10), currently operational quantum computing components (e.g., which tend to be noisy and have less than 100 operational qubits) are able to accurately determine the measured values. The classical computing component 110 may determine determined values corresponding to various inactive orbitals (e.g., core orbitals and/or virtual orbitals). The classical computing component 110 may then utilize the measured values and the determined values to determine structural, interaction, and/or other characteristics of the chemical system. For example, the classical computing entity utilizes measured values and the determined values to determine an approximation to the characteristics of the eigenstates of the total electronic Hamiltonian of the system, such as expectation values of quantum operators acting on such states, to complete the simulation of the chemical system, determine how the chemical system behaves in one or more interactions (e.g., with other chemical systems and/or with electromagnetic radiation), determine a structural characteristics of the chemical system, and/or the like.

However, given the limitations of currently operational quantum computing components, the use of a hybrid approximation when determining and/or generating a model of the chemical system does not always provide advantages in result accuracy and/or computational resources use. For example, currently operational quantum computing components may not have a sufficient number of qubits using hybrid approximations to generate and/or determine a model of a chemical system having a large number of active orbitals and/or a large number of electrons that may occupy the active orbitals. Thus, in various embodiments, a determination of which class of approximations to use to generate the model is performed. In various embodiments, the classes of approximations that the hybrid quantum-classical computing entity is configured and/or programmed to perform include hybrid approximations and, in some embodiments, classical approximations. In various embodiments, a hybrid approximation makes use of one or more measured values generated through operation of a quantum computing component of the hybrid quantum-classical computing system and a classical approximation is performed by a classical computing component without the input of measured values or any other values determined based on operation of the quantum computing component.

For example, in various embodiments, at step/operation 204, the classical computing component 110 evaluates and/or determines class selection metrics for the chemical system. In various embodiments, the class selection metrics are evaluated and/or determined based at least in part on the structure and/or characteristics of the chemical system. For example, the class selection metrics are determined based on the relative positions of one or more nuclei of the chemical system, number of electrons of the chemical system, symmetries of the chemical system, and/or the like.

In various embodiments, evaluating the class selection metrics comprises performing a multireference diagnostic of the chemical system to determine a multireference diagnostic number corresponding to the chemical system. For example, performing the multireference diagnostic of the chemical system includes determining a classical approximation of a wavefunction of the chemical system and determining a degree or measure of electron correlation of the chemical system (such as, but not limited to, the T₁ diagnostic or the D₁ diagnostic etc.) based at least in part on the classical approximation of the wave function, in an example embodiment.

In an example embodiment, the class selection metrics include one or more of a multireference diagnostic number of the chemical system, a degree or measure of electron correlation of the chemical system, a number of active orbitals of the chemical system, a number of active electrons of the chemical system, one or more symmetries of the chemical system, and/or the like.

At step/operation 206, the classical computing component 110 determines whether the class selection criteria are satisfied by the class selection metrics. In an example embodiment, the class selection criteria include one or more thresholds and/or ranges that each relate to a respective metric of the class selection metrics. To determine whether the class selection criteria are satisfied by the class selection metrics, a respective metric of the class selection metrics is compared to a respective threshold and/or range of the class selection criteria to determine whether the respective threshold and/or range is satisfied by the respective metric. For example, in an example embodiment, when it is determined that the multireference diagnostic number and/or strength of electron correlation is greater than or equal to the respective threshold strength, it is determined that the class selection metrics satisfy the class selection criteria. When it is determined that the multireference diagnostic number and/or strength of electron correlation is less than the respective threshold strength, it is determined that the class selection metrics do not satisfy the class selection criteria. For example, in an example embodiment, the multireference diagnostic number is the T₁ diagnostic and the respective threshold strength is 0.02. Various other multireference diagnostic numbers and respective threshold strengths may be used in various other embodiments, as appropriate for the application.

In an example embodiment, the one or more class selection criteria comprise a single criterium (e.g., a single threshold or range requirement). In various embodiments, the one or more class selection criteria include multiple criteria and/or use a combination of metrics (e.g., a function of the values of the class selection metrics) to determine whether the one or more class selection criteria are satisfied by the class selection metrics. In an example embodiment, at least one of the one or more class selection criteria (e.g., the thresholds and/or ranges thereof) is determined and/or set based on one or more characteristics and/or functionality of the quantum computing component 130 (e.g., number of qubits, connectivity of the qubits, and/or the like).

When it is determined, at step/operation 206, that the one or more class selection metrics do not satisfy the one or more class selection criteria, the process continues to step/operation 208. In various embodiments, at step/operation 208 the classical computing component 110 determines a model of the chemical system using a classical approximation. For example, the classical computing component 110 may be generate a model the chemical system using one or more of coupled cluster singles and doubles (CCSD), coupled cluster singles doubles (triples) (CCSD(T)), second order Moller-Plesset (MP2), or Perdew-Burke-Ernzerhof (PBE) methods.

In various embodiments, step/operation 208 may include, in addition to and/or instead of generating the model of the chemical system using the classical approximation, providing a notification that the chemical system is not a good candidate for use of a hybrid approximation in determining a model for the chemical system. For example, a notification may be provided through a user output device of the classical computing entity 110 (e.g., a display or speaker) or a communication interface of the classical computing entity 110 (e.g., an email, text message, voicemail, push notification, and/or the like). For example, the notification is configured to notify a user that the chemical system is not a good candidate for use of a hybrid approximation in determining a model for the chemical system. In an example embodiment, the notification is configured to notify a user that the chemical system is not a good candidate for use of a hybrid approximation in determining a model for the chemical system using the available quantum computing component 130.

When it is determined, at step/operation 206, that the one or more class selection metrics do satisfy the one or more class selection criteria, the process continues to step/operation 210. At step/operation 210, the classical computing component 110 evaluates and/or determines type selection metrics. In various embodiments, the type selection metrics are evaluated and/or determined based on one or more of at least a portion of a structure of the chemical system, information about a quantum computing component of the hybrid quantum-classical computing system, user input, and/or the like.

For example, in various embodiments, the second selection criteria are evaluated at least in part based on a number of active electrons of the chemical system, a number of active orbitals of the chemical system, or one or more symmetries of the chemical system, and/or the like.

In an example embodiment, the second selection criteria are evaluated at least in part based on a number of qubits of the quantum computing component 130, available run time of the quantum computing component 130, a noise profile of one or more functions of the quantum computing component 130, and/or the like.

In various embodiments, the type selection metrics are determined based on user input (e.g., received via a user input device of the classical computing component 110, for example). For example a user may provide input indicating that a particular hybrid approximation should be used to generate the model of the chemical system or provide a preference ranking of available hybrid approximations (e.g., indicting the user preference of which hybrid approximation should be used if that hybrid approximation is reasonable and/or available), and/or the like.

In various embodiments, at least a portion of the type selection metrics are evaluated and/or determined as part of the evaluation and/or determination of the class selection metrics.

At step/operation 212, the classical computing component 110 selects a hybrid approximation based on type selection criteria and the type selection metrics. For example, the type selection criteria provide a set of logic that, when evaluated based on the type selection metrics, selected and/or provides a selection of a hybrid approximation from a group of two or more hybrid approximations that the hybrid quantum-classical computing system 100 is configured and/or programmed to perform. For example, in an example embodiment, the hybrid quantum-classical computing system 100 is configured and/or programmed to generate models of chemical systems using a respectively selected one of NEVPT2, AC0, and/or one or more other hybrid approximations.

In an example embodiment, the type selection criteria is configured to cause selection of a hybrid approximation indicated by a second selection metric corresponding to user input indicating the hybrid approximation. In another example embodiment, the type selection criteria is configured to cause selection of a hybrid approximation based on an expected amount of computational resources required to generate the measured values (e.g., through operation of the quantum computing component 130) for use in generating the model using the hybrid approximation and the availability of computational resources of the quantum computing component 130. For example, classical computing component 110 may evaluate one or more functions based on values of the type selection metrics and use the resulting values of the one or more functions to select a hybrid approximation.

Once the classical computing component 110 selects the hybrid approximation, the hybrid quantum-classical computing system 100 starts generating the model of the chemical system using the selected hybrid approximation.

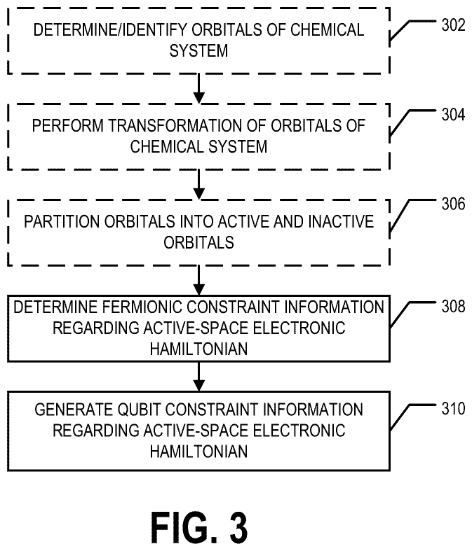

At step/operation 214, the classical computing component 110 generates qubit constraint information for the chemical system. In various embodiments, the qubit constraint information provides the information required to generate a quantum circuit or program that encodes information regarding the active-space of the chemical system. For example, in various embodiments, the qubit constraint information provides the information required to generate a quantum circuit or program that encodes the active-space electronic Hamiltonian. For example, in various embodiments, the qubit constraint information includes a translated version of the active-space electronic Hamiltonian defined in the space of two or more active orbitals in the qubit basis of the quantum computing component 130. FIG. 3 illustrates example process, procedures, and/or operations of generating qubit constraint information according to various embodiments.

In various embodiments, the qubit constraint information is generated and/or determined in a manner appropriate for the selected hybrid approximation and/or the quantum computing component 130. For example, the qubit constraint information is configured to provide the quantum computing component 130 with the information required for the quantum computing component 130 to perform a quantum circuit and/or program that results in the measurement and/or determination of measured values used by the selected hybrid approximation to generate a model of the chemical system. In various embodiments, the qubit constraint information provides the information required for the quantum computing component 130 to perform a quantum circuit and/or program that results in the measurement and/or determination of measured values used by the selected hybrid approximation to generate a model of the chemical system in a basis that is appropriate for the qubits of the quantum computing component 130.

In various embodiments, generating the qubit constraint information includes determining a first level approximation of energies and/or wavefunctions of the orbitals of the chemical system (and/or of the active-space orbitals of the chemical system). In an example embodiment, at least a portion of the information used to generate the qubit constraint information is determined at step/operation 204 (e.g., the evaluation of the class selection metrics) and/or step/operation 210 (e.g., the evaluation of the type selection metrics). In various embodiments, generating the qubit constraint information includes transforming and/or translating information corresponding to the first level approximation of the energies and/or wavefunctions of the orbitals into a qubit basis corresponding to the qubits of the quantum computing component 130.

At step/operation 216, the classical computing component 110 provides the qubit constraint information to the quantum computing component. For example, the classical computing component 110 may transmit or otherwise communicate the qubit constraint information such that the controller 132 of the quantum computing component 130 receives the qubit constraint information regarding the active-space electronic Hamiltonian.

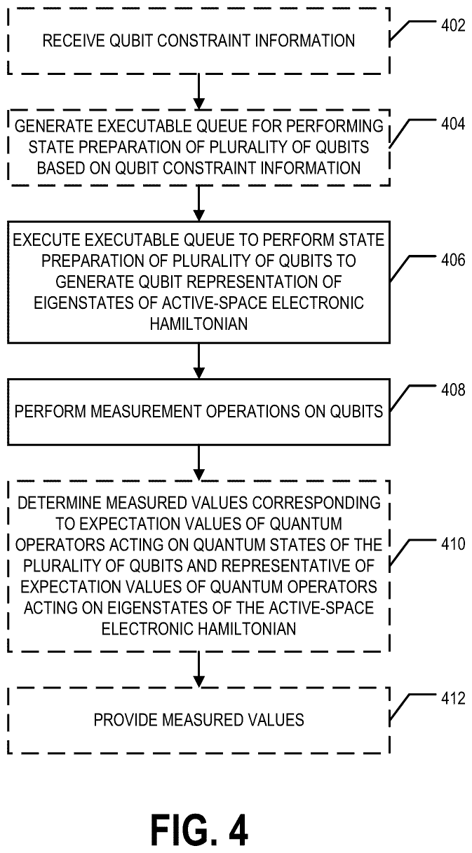

As detailed with respect to FIG. 4, the quantum computing component 130 may use the qubit constraint information to generate (e.g., through performance of a quantum circuit and/or algorithm) a qubit representation of the characteristics of the eigenstates of the active-space electronic Hamiltonian. For example, a qubit representation of a wavefunction of the active orbitals and/or eigenstates of the active-space electronic Hamiltonian may be generated through execution of a quantum circuit and/or algorithm. In various embodiments, the quantum state preparation is performed using a variational quantum eigensolver (VQE) method, variational quantum deflation, quantum subspace expansion, imaginary time evolution, variational quantum phase estimation, quantum phase estimation or qubitization, and/or the like. For example, in an example embodiment, the quantum state preparation is performed using a VQE algorithm using a unitary coupled cluster ansatz.

Various measurements may then be performed of the qubit representation to generate measured values corresponding to the active orbitals. For example, measurement operations may be performed on a plurality of qubits 134 of the quantum computing component 130 and measured values corresponding to the expectation values of quantum operators acting on at least a portion of the plurality of qubits are determined therefrom. In various embodiments, the measured values comprise at least one expectation value of the active-space electronic Hamiltonian, one or more of the spinless (i.e., spin-traced) 1-, 2-, 3-, and 4-particle reduced density matrices (RDMs), and/or the like. In various embodiments, the measured values that are measured and/or determined are determined based on the selected hybrid approximation. In various embodiments, the measured values are measured and/or determined from qubit measurements using operator averaging, partial tomography of quantum states, overlap quantum tomography, and/or the like. For example, in an example embodiment, the measured values are measured and/or determined from qubit measurements using an operator averaging method using measurement-reduction techniques.

For example, the quantum computing component 130 is configured to generate and/or measure a representation of the eigenstates of the active-space electronic Hamiltonian of the active space defined by the two or more active orbitals of the chemical system. For example, in an example embodiment, quantum computing component 130 is configured to measure one or more RDMS (e.g., one particle RDM (1-RDM), two particle RDM (2-RDM), three particle RDM (3-RDM), and/or four particle RDM (4-RDM)). Moreover, using the generated and/or measured representation of the active orbitals to generate a measurement of the one or more RDMs provides a more accurate determination of the one or more RDMs than conventional approximations to the one or more RDMs, which results in improved quantitative accuracy of the simulation of the chemical system.

At step/operation 218, the classical computing component 110 receives the measured values. For example, in various embodiments, the classical computing component 110 receives measured values corresponding to expectation values of quantum operators acting on quantum states of at least a portion of a plurality of qubits of the quantum computing component and representative of the expectation values of quantum operators acting on eigenstates of the active-space electronic Hamiltonian. For example, the quantum computing component 130 may provide the measured values such that the classical computing component 110 receives the measured values.

In various embodiments, the measured values received by the classical computing component 110 include expectation values of quantum operators acting on eigenstates of the active-space electronic Hamiltonian, spinless (i.e., spin-traced) 1-, 2-, 3-, and 4-particle RDMs, and/or the like. In an example embodiment, the measured values include spinless (i.e., spin-traced) 1-RDM and 2-RDM. In various embodiments, the classical computing component 110 generates n-RDM operators based on the measured values corresponding to the 1-RDM, 2-RDM, 3-RDM, and/or 4-RDM.

At step/operation 220 the classical computing component 110 uses the selected hybrid approximation and the measured values to generate a model of the chemical system. For example, the classical computing component 110 uses the selected hybrid approximation and the measured values to generate the model of the chemical system that represents at least one of a structural characteristic of the chemical system, a chemical interaction characteristic of the chemical system, or a response characteristic.

In various embodiments, the classical computing component 110 determines determined values corresponding to the orbitals. For example, in various embodiments, the classical computing component 110 may determine various determined values corresponding to the inactive orbitals (e.g., core orbitals and/or virtual orbitals) and/or representative of the inactive space defined by the one or more inactive orbitals. For example, the classical computing component is configured to generate and/or determine a representation of the ground and/or core orbitals of the chemical system. For example, in various embodiments, the classical computing component 110 may use various methods such, MR-MBPT, NEVPT2, AC0, second order complete active space perturbation theory (CASPT2), MR-CC, MR-CI, and/or the like to determine one or more determined values corresponding to the spatial distribution and/or energy of one or more inactive orbitals (e.g., core orbitals and/or virtual orbitals).

In various embodiments, the classical computing component 110 may determine a four particle RDM (4-RDM). For example, based on characteristics of the quantum computing component 130 and/or the chemical system, the quantum computing component 130 may not have sufficient computational resources for enabling the measuring of the 4-RDM for the active space defined by the two or more active orbitals. In such instances, the classical computing component 110 may determine the 4-RDM using, for example, a cumulant expansion technique. For example, in various embodiments, the 4-RDM is determined and/or approximated using a spinless cumulant expansion based at least in part on the 1-RDM, 2-RDM, and/or 3-RDM. This results in the computational resources (e.g., processing time/power, memory requirements) required for generating an accurate model of the chemical system to be considerably reduced. In various embodiments, the selected hybrid approximation may only require a 1-RDM and a 2-RDM and in such embodiments, the measured values and/or determined values may include the 1-RDM and 2-RDM, but not a 3-RDM and/or 4-RDM. For example, the determined values determined by the classical computing component 110 may be determined and/or controlled by the selected hybrid approximation and the information required by the selected hybrid approximation to generate the model of the chemical system.

Based on the selected hybrid approximation, the classical computing component 110 utilizes the (quantumly) measured values and the (classically) determined values to generate a model of the chemical system that is representative of at least one a structural characteristic, a chemical interaction characteristic of the chemical system, or a response characteristic of the chemical system. In various embodiments, the classical computing component 110 utilizes the measured values representative of the expectation values of the quantum operators acting on the eigenstates of the active-space electronic Hamiltonian to yield an approximation to expectation values of quantum operators acting on eigenstates of the total electronic Hamiltonian to generate a model of the chemical system that represents at least one of a structural characteristic of the chemical system, a chemical interaction characteristic of the chemical system, or a response characteristic of the chemical system. For example, the at least one of an expectation value for the active-space electronic Hamiltonian or at least one RDM is used to generate a model of the chemical system.

In an example embodiment, the hybrid quantum-classical computing system 100 is configured and/or programmed to generate a model of the chemical system using a strongly-contracted NEVPT2 calculation. For example, in an example embodiment, the selected hybrid approximation is selected from a group of hybrid approximations that includes a strongly-contracted NEVPT2 approximation. For example, the classical computing component 110 may use the determined and measured values to perform a strongly-contracted NEVPT2 calculation to determine corrections to the energies and/or wavefunctions of the orbitals determined and/or identified at step/operation 214. In an example embodiment, a spinless formulation of strongly-contracted NEVPT2 technique is used to determine the corrections to the approximate energy and/or wavefunction of the orbitals determined and/or identified at step/operation 214 using the measured values (e.g., expectation value for the active-space electronic Hamiltonian, 1-RDM, 2-RDM, 3-RDM, and/or 4-RDM). The corrections may then be applied to the energies and/or wavefunctions of the orbitals to generate a model of the chemical system that represents a structural characteristic of the chemical system, a chemical interaction characteristic of the chemical system, a response characteristic of the chemical system, and/or the like.

In an example embodiment, the hybrid quantum-classical computing system 100 is configured and/or programmed to generate a model of the chemical system using an adiabatic connection (AC) technique with the first-order expansion of the AC integrand at the coupling constant equal to 0 (AC0). For example, in an example embodiment, the selected hybrid approximation is selected from a group of hybrid approximations that includes an AC0 approximation. For example, the classical computing component 110 may use the determined and measured values to perform an AC0 calculation to determine corrections to the energies and/or wavefunctions of the orbitals determined and/or identified at step/operation 214. In an example embodiment, an AC0 technique is used to determine the corrections to the approximate energy and/or wavefunction of the orbitals determined and/or identified at step/operation 214 using the measured values (e.g., expectation value for the active-space electronic Hamiltonian, 1-RDM and/or 2-RDM). The corrections may then be applied to the energies and/or wavefunctions of the orbitals to generate a model of the chemical system that represents a structural characteristic of the chemical system, a chemical interaction characteristic of the chemical system, a response characteristic of the chemical system, and/or the like.

In various embodiments, a structural characteristic of the chemical system characterizes and/or provides information regarding the structure of the chemical system. For example, the structural characteristic may indicate a shape of the chemical system, an ordering and/or spatial distribution of the component atoms or atom groups within the chemical system, a characterization of various bonds of the chemical system, relative nuclei positions within the chemical system, and/or the like.

In various embodiments, a chemical interaction characteristic of the chemical system characterizes and/or provides information regarding how the chemical system interacts with one or more other chemical systems of the same or different types. For example, the chemical interaction characteristic of the chemical system may provide information regarding how the chemical system interacts with other chemical systems of the same type/chemical formula as the chemical system or of different types/chemical formulas than that of the chemical system.

For example, the structural and/or chemical interaction characteristic may be a dissociation curve, bond energy, reaction energy, reaction barrier, binding energy, adsorption energy and/or the like.

In various embodiments, the response characteristics of the chemical system characterize and/or provide information regarding how the chemical system interacts with electromagnetic radiation. For example, a response characteristic of the chemical system may be an excitation energy, singlet-triplet gap, photodissociation energy, photoionization energy, absorption cross-section, dielectric constant, dielectric function, oscillator strength and/or the like.

At step/operation 222, the classical computing component 110 provides at least a portion of the model of the chemical system representing the structural characteristic, the chemical interaction characteristic of the chemical system, and/or the response characteristic of the chemical system. In various embodiments, providing the at least a portion of the model of the chemical system comprises displaying, storing, transmitting (e.g., via one or more wired and/or wireless networks), providing a call response (e.g., application program interface (API) call response), and/or the like.

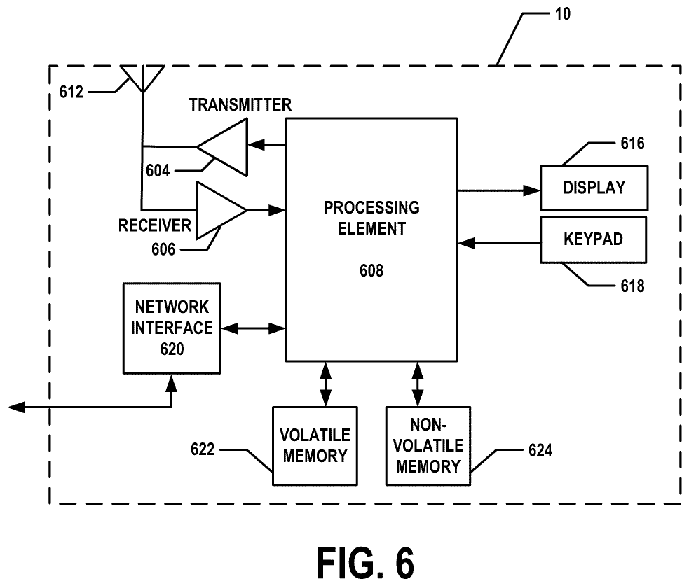

For example, in an example embodiment, the classical computing component 110 causes a display (e.g., display 616 illustrated in FIG. 6 and/or another display) to display a representation of the model of the chemical system. For example, a graphics processing unit (GPU) of the classical computing component 110 may generate a graphical representation of at least a portion of the model of the chemical system that, for example, provides a visualization of the structural characteristic, chemical interaction characteristic of the chemical system, and/or response characteristic of the chemical system. The classical computing component 110 may then cause the graphical representation of the at least a portion of the model of the chemical system to be displayed via a display for review and/or viewing by a human user.

In another example, the classical computing component 110 may generate and store (e.g., in memory 622, 624) a file comprising at least a portion of the model of the chemical system. For example, the file may comprise the corrected energies and/or wavefunctions of the orbitals, the structural characteristic, the chemical interaction characteristic, the response characteristic, the chemical formula of the chemical system, and/or the like. The file may then be provided to one or more programs, applications, modules, and/or the like operating on the classical computing component 110 or another computing entity as input for one or more functions and/or computations performed thereby. For example, the file storing and/or encoding the at least a portion of the model of the chemical system may be used by various programs, applications, modules, and/or the like to generate graphical representations and/or visualizations of the chemical system and/or portions thereof, perform simulations that include the interaction of the chemical system with one or more other chemical systems (of the same of different chemical formulas), perform simulations of a bulk material that includes the chemical system, perform simulations that include the interaction of the chemical system with one or more biological systems, and/or the like.

FIG. 3 provides a flowchart illustrating various processes, procedures, operations, and/or the like performed by a classical computing component 110 to perform the classical pre-processing to generate qubit constraint information. For example, the classical computing component 110 may perform the steps, processes, procedures, operations, and/or the like shown in FIG. 3 to generate the qubit constraint information, in various embodiments. For example, the processes, procedures, operations, and/or the like shown in FIG. 3 may be performed as part of step/operation 214, in various embodiments.

Starting at step/operation 302 of FIG. 3, the classical computing component 110 determines and/or identifies approximations to the electron states of the chemical system. The approximations to the electron states of the chemical system are determined and/or identified by generating and/or determining approximations to the respective wavefunctions corresponding to the electron states. For example, in an example embodiment, the classical computing component 110 generates an approximation of the wavefunctions and energies of the electron states of the chemical system using a quantum many-body system approximation. For example, each wavefunction corresponds to one of the electron states of the chemical system.

For example, in various embodiments, the classical computing component 110 performs a first level simulation of the chemical system to identify and/or determine approximations to the orbitals of the chemical system. For example, the classical computing component 110 may use a mean-field approximation method such as the Hartree-Fock theory where the electronic wavefunction is defined by a single Slater determinant, yielding, inter alia, molecular orbitals, to identify and/or determine approximations to the orbitals of the chemical system. In various embodiments, the classical computing component 110 constructs a more accurate approximation where said orbitals can be rotated and split into active and inactive orbitals, and the electronic wavefunction is defined by a linear combination of Slater determinants constructed by different occupations of said active orbitals. The inactive orbitals include the core and virtual orbitals. However, as one of ordinary skill in the art would understand, the generated and/or determined approximations to the wavefunctions and energies are first order approximations that are not sufficiently accurate enough to alone provide an effective model of the chemical system.

In various embodiments, performance of step/operation 302 is performed as part of evaluating and/or determining the class selection metrics at step/operation 204 and/or evaluating and/or determining the type selection metrics at step/operation 210. In such embodiments, the information regarding the orbitals of the chemical system may be accessed from memory (e.g., cache) during the generation of the qubit constraint information.

At step/operation 304, the classical computing component 110 may transform the respective orbitals of the chemical system. For example, the classical computing component 110 may determine respective localizations of the respective orbitals. For example, the wavefunctions may be spatially and/or energetically localized, based at least in part on the structural and/or chemical interaction characteristic of interest. For example, if a particular bond of the chemical system is of particular interest, one or more appropriate orbitals may be localized to the area of the particular bond. In various embodiments, the transformation of an orbital is performed by performing a rotation of the corresponding wavefunction into a desired coordinate system and/or the like.

At step/operation 306, the classical computing component 110 partitions the orbitals of the chemical system into active orbitals and inactive orbitals (e.g., including core orbitals, virtual orbitals, and/or the like). For example, the classical computing component 110 may process the generated and/or determined wavefunctions and/or the energies associated therewith and identify and/or select the active orbitals therefrom.

The active orbitals define an active space of the Hilbert space of the chemical system and the inactive orbitals define an inactive space of the Hilbert system of the chemical system. In other words, the active space comprises and/or consists of the active orbitals and the inactive space comprises and/or consists of the inactive orbitals.

In various embodiments, the core orbitals are the orbitals of the chemical system that, in the reference mean-field (Hartree-Fock) wavefunction are occupied by two electrons and have not been chosen to be active orbitals, for example due to their negligible interaction with other active orbitals. In various embodiments, the virtual orbitals comprise orbitals which are unoccupied in the reference mean-field (Hartree-Fock) wavefunction and have not been chosen to be active orbitals.

In various embodiments, the active orbitals are identified and/or selected based on user input (e.g., received via a user input device of the classical computing component 110). In various embodiments, the active orbitals are identified and/or selected based at least in part on the structural characteristic, chemical interaction characteristic, and/or response characteristic that the model of the chemical system is to represent. In various embodiments, the active orbitals include and/or consist of a selection of the orbitals that were occupied in the approximations to the orbitals that were determined as part of step/operation 302 (e.g., the reference Hartree-Fock wavefunction for the chemical system) and a selection of the orbitals that were not occupied in the approximation of the electron states that were determined as part of step/operation 302.

In various embodiments, two or more orbitals are selected and/or identified as active orbitals. For example, the active space is defined based on two or more active orbitals. In various embodiments, two to six orbitals are selected and/or identified as active orbitals. In various embodiments, up to a hundred orbitals are selected and/or identified as active orbitals. In various embodiments, the number and/or maximum number of orbitals selected and/or identified as active orbitals is determined based on the number of qubits 134 of the quantum computing component 130 of the hybrid quantum-classical computing system 100.

In an example embodiment, the partitioning of the orbitals into active and inactive orbitals is performed automatically by the classical computing component 110 (e.g., based on execution of computer-readable instructions without human user interaction). In an example embodiment, the partitioning of the orbitals into active and inactive orbitals is performed through user interaction with the classical computing component (e.g., based on user input received via one or more user input devices of the classical computing component 110).

At step/operation 308, the classical computing component 110 determines fermionic constraint information regarding the active space. In various embodiments, the fermionic constraint information comprises an effective fermionic Hamiltonian of the active space. In an example embodiment, the fermionic constraint information comprises an active-space electronic Hamiltonian defined in the space of two or more active orbitals (e.g., in defined in the active space). In an example embodiment, the fermionic constraint information is a description of the energy and/or wavefunction of the electrons in the active orbitals. For example, in an example embodiment, the fermionic constraint information comprises an operator configured to operate on the wavefunctions corresponding to the active orbitals to provide energy information corresponding to the active orbitals and/or the wavefunction that the operator acted upon. For example, in an example embodiment, the fermionic constraint information comprises a Hamiltonian of the active orbitals. In various embodiments, the fermionic constraint information corresponds to the active space defined by the two or more active orbitals. For example, the fermionic constraint information does not correspond to the inactive space defined by the one or more inactive orbitals.

In various embodiments, the fermionic constraint information is determined using a procedure known as complete active space configuration interaction (CAS-CI) or a selected active space configuration interaction corresponding to a full configuration interaction (CFI) calculation restricted to the two or more active orbitals. For example, a wavefunction is expressed as a linear combination of all or a subset of configurations obtained by exciting the electrons within the active space while the core orbitals remain completely full and the virtual orbitals remain empty. For example, for a chemical system having two active orbitals |A> and |B>, the wavefunction is expressed in the form a|A>+b|B>, where |a²+b²|=1.

In an example embodiment, the fermionic constraint information is determined using a multi-configurational self-consistent field (MC-SCF) technique such that the wavefunction is expressed as a linear combination of all or a selection of configurations obtained by exciting the electrons within the active space while the core orbitals remain completely full and the virtual orbitals remain empty is optimized to minimize the active space configuration interaction (CI) energy.

In various embodiments, the fermionic constraint information is determined using a multi-reference technique that corrects and/or adds dynamical correlations to active space (AS) electron wavefunction determination. For example, in an example embodiment, the fermionic constraint information is determined using a multi-reference configuration interaction (MR-CI) technique where the wavefunctions comprise linear combinations of electron configurations constructed by applying excitation operators to a AS wavefunction.

In an example embodiment, the fermionic constraint information is determined using a multi-reference many-body perturbation theory (MR-MBPT) technique. For example, perturbation theory techniques, such as the Moller-Plesset method (e.g., second order Moller-Plesset method), may be applied to a CAS Hamiltonian to determine fermionic constraint information.

In an example embodiment, a second-order N-electron valence state perturbation theory (NEVPT2) technique is used to determine the fermionic constraint information. For example, the two electron Dyall Hamiltonian may be used as a starting point for applying the excitation operator and determining the configurations for inclusion in the electron wavefunction included and/or used at least in part to determine the fermionic constraint information.

In an example embodiment, a multi-reference coupled cluster theory (MR-CC) is used to determine the fermionic constraint information.

In an example embodiment, an adiabatic connection (AC) technique with the first-order expansion of the AC integrand at the coupling constant equal to 0 (AC0) is used to determine the fermionic constraint information.

In various embodiments, determining the fermionic constraint information comprises determining the active-space electronic Hamiltonian. In various embodiments, the active-space electronic Hamiltonian is determined and/or defined based at least in part on the one electron effective Hamiltonian for the active orbitals, determined two electron integrals, and the nuclear repulsion energy for the chemical system, which are determined as part of steps/operations 302 and 304, in various embodiments. For example, the effective one electron Hamiltonian and a transformed two electron integral in the molecular orbital basis set are generated and used to define the active-space electronic Hamiltonian.

At step/operation 310, the classical computing component 110 generates qubit constraint information regarding the active orbitals by transforming and/or translating the fermionic constraint information into a qubit basis. The qubit basis is determined based on the quantum computing component 130 to be used to determine the measured values corresponding to the active orbitals. For example, the qubit basis for a quantum computing component 130 that uses trapped-ion qubits is different from the qubit basis for a quantum computing component 130 that uses Josephson junction qubits.

In an example embodiment, the qubit constraint information comprises a active-space electronic Hamiltonian mapped, translated, and/or transformed into the qubit basis to provide a qubit Hamiltonian encoding the spatial and energy constraints of the active-space electronic Hamiltonian. For example, classical computing component 110 translates, maps, and/or transforms the fermionic constraint information regarding the active-space electronic Hamiltonian into a qubit basis to generate qubit constraint information regarding the active-space electronic Hamiltonian.

In various embodiments, the qubit basis corresponds to qubit operators that may be performed on the qubits of the quantum computing component 130. Thus, mapping, translating, and/or transforming the fermionic constraint information into the qubit constraint information comprises translating or mapping the fermionic operators of the active-space electronic Hamiltonian of the fermionic constraint information into the qubit operators of the qubit basis. In various embodiments, a Jordan-Wigner, Bravyi-Kitaev, Z₂-symmetries, low density parity check (LDPC), segment, CI-matrix, degree-D, optimal-degree, and/or other mapping or encoding for transforming the fermionic constraint information into qubit constraint information.

In various embodiments, the qubit constraint information comprises ansatz information. For example, the ansatz information may be used to formulate the representations of the wavefunctions that were mapped and/or translated into the qubit basis. For example, the ansatz may be a predicted functional form of the wavefunctions corresponding to the active orbitals, in various embodiments. In an example embodiment, the ansatz is a symmetry-adapted singlet unitary coupled-cluster singles and doubles (UCCSD) ansatz.

FIG. 4 provides a flowchart illustrating various processes, procedures, operations, and/or the like performed by the quantum component of the hybrid quantum-classical computing system 100 to generate and/or provide a model of the chemical system that represents structural characteristics of the chemical system, chemical interaction characteristics of the chemical system, response characteristic of the chemical system, and/or the like. In various embodiments, steps/operations 402-412 are performed between performance of steps/operations 216 and 218.

Starting at step/operation 402, the controller 132 of the quantum computing component 130 receives the qubit constraint information. For example, the qubit constraint information is the mapping and/or translation of the fermionic constraint information into a qubit basis corresponding to the quantum computing component 130. For example, the qubit constraint information includes a translated version of the active-space electronic Hamiltonian defined in the space of two or more active orbitals in the qubit basis of the quantum computing component 130.

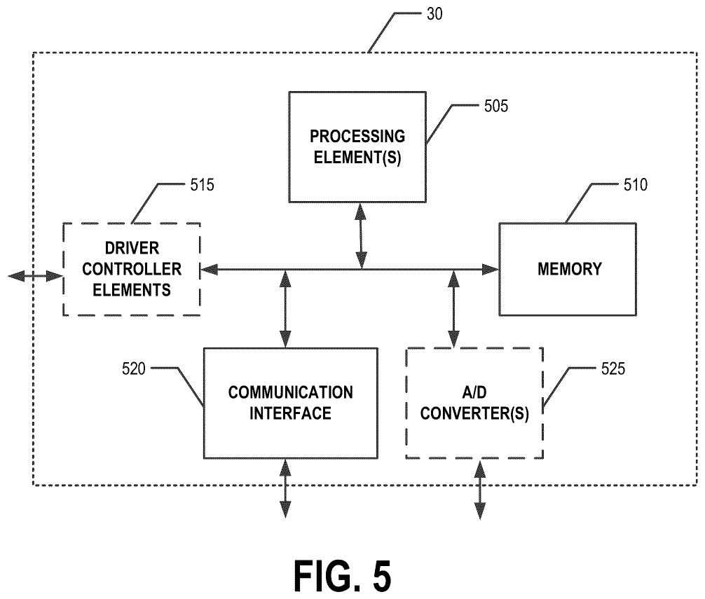

In various embodiments, the controller 132 receives the qubit constraint information via communications interface 520 (see FIG. 5). For example, the classical computing component 110 may provide the qubit constraint information such that the quantum computing component 130 receives the qubit constraint information.

At step/operation 404, the controller 132 of the quantum computing component generates and/or determines an executable queue of commands for performing state preparation of a plurality of qubits 134 based on the qubit constraint information. For example, a quantum circuit and/or algorithm is defined, determined, generated, and/or compiled for performing state preparation of the plurality of qubits 134 such that the quantum states of the qubits 134, after the performance of the quantum circuit and/or algorithm, are representative of wavefunctions and/or energies of the active orbitals. For example, the quantum circuit and/or algorithm is defined, determined, generated, and/or compiled for performing state preparation of the plurality of qubits 134 such that the quantum states of the qubits 134, after the performance of the quantum circuit and/or algorithm, are representative of eigenstates of the active-space electronic Hamiltonian. For example, the occupations of N active orbitals (fermionic modes) are mapped to N (or close to N) qubits 134 via the quantum circuit and/or algorithm.

For example, the quantum circuit and/or algorithm may include an ordered combination of single and two or more qubit gates performed on particular qubits 134 such that the quantum states of the qubits a representative of wavefunctions and/or energies of the active orbitals of the chemical system and/or representative of eigenstates of the active-space electronic Hamiltonian. The ordered combination of single and two or more qubit gates is determined based on the qubit constraint information. In an example embodiment, the quantum circuit and/or algorithm is defined, determined, generated, and/or compiled using a VQE algorithm using a unitary coupled cluster ansatz. In various embodiments, various other ansatzes may be used.

In various embodiments, measurement reduction techniques are used to partition the quantum circuit and/or algorithm into commuting sets so as to reduce a number of measurements required.

Continuing with FIG. 4, at step/operation 406, the controller 132 executes the executable queue to perform the state preparation of the qubits 134. For example, the controller 132 executes the executable queue to cause the qubit manipulation elements 136 to cause performance of the ordered combination of single and two or more qubit gates to cause the quantum states of the qubits 134 to represent the wavefunctions and/or energies of the active orbitals of the chemical system and/or eigenstates of the active-space electronic Hamiltonian.

At step/operation 408, the controller 132 causes measurement operations to be performed to determine quantum states of the qubits 134. For example, the controller 132 controls operation of the qubit manipulation elements 136 and monitors signals received from the sensors 138 to determine respective quantum states of the qubits 134. In an example embodiment, a symmetry check is performed (e.g., by the controller 132 and/or classical computing component 110) to determine which elements are vanishing and therefore need not be measured. For example, in an example scenario, the number of active electrons equal to two causes the 3-RDM and 4-RDM to vanish (e.g., be substantially equal to zero). Thus, in such an example, computing resources may be saved by determining that the 3-RDM and 4-RDM need not be measured. In another scenario, the symmetry of the active space Hamiltonian may cause some matrix elements of the 1-RDM, 2-RDM, 3-RDM and/or 4-RDM to vanish (e.g., be substantially equal to zero). Thus, in such an example, computing resources may be saved by determining that said matrix elements of the 1-RDM, 2-RDM, 3-RDM and/or 4-RDM need not be measured.

In various embodiments, the state preparation performed at step/operation 406 and the measurement operations performed at step/operation 408 are determined based on the selected hybrid approximation. For example, when the selected hybrid approximation is NEVPT2, measurements corresponding to one or more matrix elements of each of the 1-RDM, 2-RDM, 3-RDM and/or 4-RDM are determined, and, when the selected hybrid approximation is AC0, measurements corresponding to one or more matrix elements of each of the 1-RDM, 2-RDM (but no matrix elements corresponding to the 3-RDM and/or 4-RDM) are determined, in an example embodiment.

At step/operation 410, the controller 132 determines measured values for the corresponding to the active orbitals based on the results of the measurement operations (e.g., the measured quantum states of the qubits 134). For example, in various embodiments, the measured values comprise expectation values of the active space Hamiltonian, spinless (i.e., spin-traced) 1-, 2-, 3-, and 4-particle reduced density matrices (RDMs), and/or the like. For example, in various embodiments, the measured values correspond to expectation values of quantum operators acting on quantum states of at least a portion of a plurality of qubits of the quantum computing component 130 and/or are representative of the expectation values of quantum operators acting on eigenstates of the active-space electronic Hamiltonian. In various embodiments, the measured values are measured and/or determined from qubit measurements using operator averaging, partial tomography of quantum states, overlap quantum tomography, and/or the like. For example, in an example embodiment, the measured values are measured and/or determined from qubit measurements using an operator averaging method using measurement-reduction techniques.

At step/operation 412, the controller 132 causes the quantum computing component 130 (e.g., via communication interface 520) to provide the measured values. For example, the quantum computing component 130 provides the measured values such that the classical computing component 110 receives the measured values and determines and/or generates the model of the chemical system based at least in part thereon. For example, the quantum computing component 130 may provide the measured values for receipt by the classical computing component 110 via one or more wired and/or wireless networks 120, directed wired and/or wireless communication, and/or the like.

As illustrated in FIG. 4, the controller 132 determines the measured values based on the results of the measurement operations and provides the measured values for receipt by the classical computing component 110. In an example embodiment, the controller 132 provides the results of the measurement operations for receipt by the classical computing component 110 and the classical computing component 110 determines the measured values based on the results of the measurement operations.

### Technical Advantages

Conventional classical computer software products are known that can be executed on classical computing hardware, for example on classical non-quantum computing components, to simulate chemical systems and to determine their manners of interactions with other molecules, electromagnetic fields and electromagnetic radiation. Such computer software products are configured to compute approximations to the electronic states of the chemical system, for example by means of the Hartree-Fock theory where the electronic wavefunction is defined by a single Slater determinant, or Density Functional Theory, where the electronic density is computed by a single Slater determinant, and then compute other approximations to the electronic states of the system, including single-reference, active-space and multireference (multiconfigurational) approximations, as a second category of computation to take into account electronic correlation effects.

An objective technical problem that is encountered in practice is that computing resources required for implementing the second category of computations can be very challenging; in certain situations, the amount of computing resources required can become intractable. As a result, approximations are conventionally often used when performing the computations associated with the aforesaid second category. In various scenarios, these approximations are not sufficiently accurate to provide structural and/or interaction characteristics of the atom or molecule to account for and/or predict real world observations of the atom or molecule and/or interactions thereof.

Quantum computing components are expected to provide systems that can perform complex computations in shortened time frames. However, currently operational quantum computing components tend to include relatively low numbers of qubits (e.g., less than 100 qubits) and tend to be relatively noisy. As a result, a further technical problem exists in that currently operational quantum computing components do not provide sufficient computational resources for performing complete chemical system simulations or even merely simulations of the excited states of a chemical system.

Various embodiments provide technical solutions to these technical problems. For example, in various embodiments, measured values for model active-space Hamiltonian orbitals are determined using the quantum component of the hybrid quantum-classical computing system. Many chemical systems, called Multi-Reference systems, cannot be adequately modelled by single-reference wavefunctions, where only one Slater determinant (electronic configuration), called the Reference Determinant, has a large coefficient in the Configuration Interaction expansion. The electronic structure of such systems is typically modelled by Full Configuration Interaction, where all orbitals are considered active and all Slater determinants obtained by promotion of electrons to virtual orbitals are included; or by Multi-Reference methods, where a subset of orbitals and electrons are considered active and a multi-configurational Reference Wavefunction is constructed as a linear combination of Slater determinants obtained by all possible occupations of active electrons in active orbitals. Said multi-configurational Reference Wavefunction is subsequently further refined, by application of Multi-Reference Configuration Interaction, Multi-Reference Perturbation Theory or Multi-Reference Coupled Cluster method, to allow for excitation of electrons into virtual orbitals outside of the active space and calculation of the dynamic correlation energy. Determination of the Reference Wavefunction is thus equivalent to a Full Configuration Interaction calculation on the active space. Because of the combinatorial (with respect to the number of active electrons and active orbitals) computational cost of the determination of coefficients in the Reference Wavefunction on a classical computer, classical implementations of Multi-Reference methods are typically limited to active spaces consisting of up to about 20 electrons in 20 orbitals. However, the subsequent calculation of dynamic correlation energy, especially by means of Configuration Interaction or Perturbation Theory, can be efficiently performed classically (i.e. in polynomial time). A quantum implementation of the Full Configuration Interaction simulation would require mapping of all orbitals to a qubit register. However, a hybrid quantum-classical implementation of a Multi-Reference method can be constructed by limiting the problem passed to the quantum component to the active electrons and active orbitals. Thus, the computational power of the quantum computing component can be used only on the classically-hard part of the electronic structure problem, where it can have an advantage over a classical algorithm, instead of being applied to the entirety of the electronic structure problem, which consists of subproblems that have a classically-efficient solution.

For example, various embodiments are configured to provide improved models of chemical systems that improve the operating of programs, applications, and/or modules that use models of chemical systems for perform various tasks (simulate chemical interactions, bulk material properties of material comprising chemical systems, interactions of chemical systems with biological systems, interactions of materials with electromagnetic fields and/or waves, and/or the like). Moreover, various embodiments, provide improvements over the functioning of classical computers through the use of a hybrid quantum-classical computing system that harnesses the currently available computational abilities of currently functional quantum computing components to generate measured values corresponding to the active orbitals of chemical systems of interest. This use of the quantum computing capabilities supplements and/or expands the ability of the computing system to generate accurate representations of chemical systems, while overcoming technical problems relating to the relatively small numbers of qubits available in currently functional quantum computing components. Thus, various embodiments provide practical applications that provide improved computer system functioning which results in improved chemical system modeling.

Additionally, in various embodiments, prior to using quantum computing resources, a check is performed to determine whether the chemical system is a good candidate for the determination of a hybrid quantum-classical approximation model. For example, class selection metrics are determined based on structural characteristics of the chemical system and compared to class selection criteria to determine whether the chemical system is a good candidate for the determination of a hybrid quantum-classical approximation model. For example, when the class selection metrics satisfy the class selection criteria, it is expected that the use of a quantum computing component in the determination of the model of the chemical system will provide a more accurate and/or computationally efficient result. When the class selection metrics do not satisfy the class selection criteria, it is expected that the use of a quantum computing component in the determination of the model of the chemical system is not likely to provide an advantage and therefor, quantum computing resources may not be used to determine the model of the chemical system. This provides the advantage of ensuring the (limited) quantum computing resources (e.g., the quantum computing component of the hybrid quantum-classical computing system) are used efficiently and effectively, resulting in improved performance of the hybrid quantum-classical computing system.

Moreover, in various embodiments, the hybrid quantum-classical computing system is configured to select a hybrid approximation to be used in generating a model of a chemical system. For example, the hybrid quantum-classical computing system is configured to use two or more hybrid approximations in generating a model of a chemical system. The classical computing entity may evaluate type selection metrics and compare the type selection metrics to approximation selection criteria to select a hybrid approximation to be used in generating the model of the chemical system. In various embodiments, the type selection metrics are determined based on one or more of at least a portion of an atomic structure of the chemical system, information about a quantum computing component of the hybrid quantum-classical computing system, user input, and/or the like. Thus, a hybrid approximation that efficiently and effectively uses the (limited) quantum computing resources (e.g., the quantum computing component of the hybrid quantum-classical computing system) can be selected for use. This functionality of various embodiments results in improved performance of the hybrid quantum-classical computing system and improved user experience.

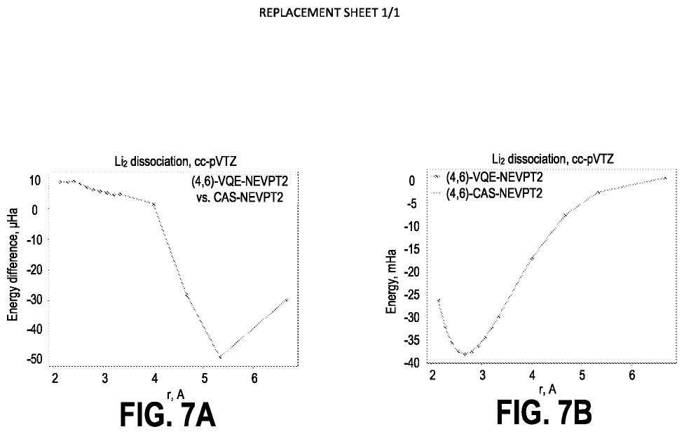

For example, an example embodiment is used to determine a dissociation curve of the dilithium molecule (Li₂). Such a simulation is a prototypical multi-reference problem, that is poorly described by mean-field methods or common single-reference correlated methods (such as second order Moller-Plesset (MP2) methods or coupled cluster singles and doubles (CCSD) methods). For the illustrated calculations, a correlation-consistent polarized valence only triple-zeta (cc-pVTZ) basis set is beneficially used. The NEVPT2 calculations were performed using an active space consisting of 4 electrons in 6 active spatial orbitals (12 spin-orbitals), which can be optimized with a complete active space self-consistent field (CASSCF) technique. In the VQE-NEVPT2, the 12 spin-orbitals are susceptible to being mapped into 12 qubits via Jordan-Wigner mapping. The ansatz used is beneficially a symmetry-adapted singlet UCCSD. FIGS. 7A and 7B provide a graphical representation of the model of the chemical system and a comparison thereof with previously determined Li₂ dissociation curves. For example, FIG. 7A illustrates the deviation of the Li₂ dissociation curve determined in accordance with an example embodiment, from conventional CAS-NEVPT2 results and FIG. 7B illustrates the Li₂ dissociation curve determined in accordance with an example embodiment together with one determined with conventional CAS-NEVPT2 results. Thus, as shown by FIGS. 7A and 7B, various embodiments provide structural characteristics, chemical interaction characteristics, and/or response characteristics of chemical systems that are consistent with existing results for simple systems and that provide more efficient use of computational resources than conventional techniques.

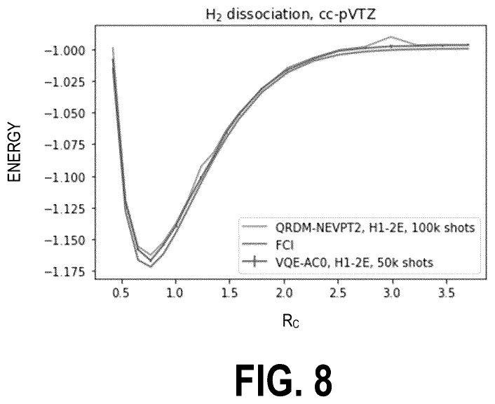

Another example embodiment is used to determine the dissociation curve of the dihydrogen molecule (H₂). FIG. 8 illustrates three determined and/or generated H₂ dissociation curves determined using a NEVPT2 hybrid approximation, a VQE-AC0 hybrid approximation, and a classical full configuration interaction (FCI) approximation. Various embodiments provide the advantage that a user is able to double check a first hybrid approximation with a second (or additional) hybrid approximation for confirmation of model predictions. As shown in FIG. 8, illustrates the various embodiments provide structural characteristics, chemical interaction characteristics, and/or response characteristics of chemical systems that are consistent with existing results for simple systems and that provide more efficient use of computational resources than conventional techniques.

### Exemplary Controller

In various embodiments, hybrid quantum-classical computing system 100 comprises a quantum computing component 130. The quantum computing component 130 is configured to perform various quantum computations and/or calculations via execution of one or more quantum circuits and/or algorithms. In various embodiments, the quantum computing component 130 configured to control operation of one or more components of the quantum computing component 130 (e.g., qubit manipulation elements 136, sensors 138), receive sensor signals indicating measurements captured by sensors 138, and/or communicate with a classical computing component 110.

As shown in FIG. 5, in various embodiments, the controller 132 may comprise various controller elements including processing elements 505, memory 510, driver controller elements 515, a communication interface 520, analog-digital converter elements 525, and/or the like. For example, the processing elements 505 may comprise one or more processing devices such as programmable logic devices (CPLDs), microprocessors, coprocessing entities, application-specific instruction-set processors (ASIPs), integrated circuits, application specific integrated circuits (ASICs), field programmable gate arrays (FPGAs), programmable logic arrays (PLAs), hardware accelerators, other processing devices and/or circuitry, and/or the like. The term circuitry may refer to an entirely hardware embodiment or a combination of hardware and computer program products. In an example embodiment, the processing element 505 of the controller 132 comprises a clock and/or is in communication with a clock.

For example, the memory 510 may comprise non-transitory memory such as volatile and/or non-volatile memory storage such as one or more of as hard disks, ROM, PROM, EPROM, EEPROM, flash memory, MMCs, SD memory cards, Memory Sticks, CBRAM, PRAM, FeRAM, RRAM, SONOS, racetrack memory, RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. In various embodiments, the memory 510 may store a queue of commands to be executed to cause a quantum algorithm and/or circuit to be executed (e.g., an executable queue), qubit records corresponding the qubits of quantum computing component (e.g., in a qubit record data store, qubit record database, qubit record table, and/or the like), a calibration table, computer program code (e.g., in a one or more computer languages, specialized controller language(s), and/or the like), and/or the like. In an example embodiment, execution of at least a portion of the computer program code stored in the memory 510 (e.g., by a processing element 505) causes the controller 132 to perform one or more steps, operations, processes, procedures and/or the like described herein for controlling operation of one or more qubit manipulation elements 136, processing sensor signals indicating measurements captured by sensors 138, and/or communicating with a classical computing component 110 of the hybrid quantum-classical computing system 100.

In various embodiments, the driver controller elements 510 may include one or more drivers and/or controller elements each configured to control one or more drivers. In various embodiments, the driver controller elements 510 may comprise drivers and/or driver controllers. For example, the driver controllers may be configured to cause one or more corresponding drivers to be operated in accordance with executable instructions, commands, and/or the like scheduled and executed by the controller 132 (e.g., by the processing element 505). In various embodiments, the driver controller elements 515 may enable the controller 132 to operate various ones of the qubit manipulation elements 136 and/or sensors 138. In various embodiments, the drivers may comprise laser drivers configured to operate one or lasers; drivers for controlling operation of one or more voltage/current sources to cause generation and providing of one or more voltage and/or current signals; and/or various other drivers configured to control operation of respective qubit manipulation elements 136 of the quantum computing component 130.

In various embodiments, the controller 132 comprises means for communicating and/or receiving signals from one or more sensors (e.g., photodetectors, voltage/current sensors, temperature sensors, pressure sensors, and/or other sensors). For example, the controller 132 may comprise one or more analog-digital converter elements 525 configured to receive signals from one or more sensors.

In various embodiments, the controller 132 comprises a communication interface 520 for interfacing and/or communicating with a classical computing component 110 of the hybrid quantum-classical computing system 100. For example, the controller 132 may comprise a communication interface 520 for receiving qubit constraint information, executable instructions, command sets, and/or the like from the classical computing component 110 and providing output received from the quantum computing component 130 (e.g., via sensors 138) and/or the result of a processing the output to determine measured values corresponding to the active orbitals to the classical computing component 110. In various embodiments, the classical computing component 110 and the controller 132 may communicate via a direct wired and/or wireless connection and/or via one or more wired and/or wireless networks 120.

### Exemplary Classical Computing Component

FIG. 6 provides an illustrative schematic representative of an example computing entity 10 that can be used in conjunction with embodiments of the present invention. In various embodiments, a classical computing component 110 is configured to interface with a quantum computing component 130. For example, the classical computing component 110 is configured to interface with a quantum computing component 130 so as to enable the efficient and accurate modeling of a chemical system through the direct modeling of aspects of the active orbitals using the quantum computing component 130. For example, the classical computing component 110 may be configured to communicate with the quantum computing component 130 allow a user (e.g., a human user or a program operating on the classical computing component 110) to provide input to the quantum computing component 130 and receive, display, analyze, and/or the like output from the quantum computing component 130.

As shown in FIG. 6, a classical computing component 110 can include an antenna 612, a transmitter 604 (e.g., radio), a receiver 606 (e.g., radio), and a processing element 608 that provides signals to and receives signals from the transmitter 604 and receiver 606, respectively. The signals provided to and received from the transmitter 604 and the receiver 606, respectively, may include signaling information/data in accordance with an air interface standard of applicable wireless systems to communicate with various entities, such as a controller 132, other classical computing entities 110, and/or the like. In this regard, the classical computing component 110 may be capable of operating with one or more air interface standards, communication protocols, modulation types, and access types.

For example, the classical computing component 110 may be configured to receive and/or provide communications using a wired data transmission protocol, such as fiber distributed data interface (FDDI), digital subscriber line (DSL), Ethernet, asynchronous transfer mode (ATM), frame relay, data over cable service interface specification (DOCSIS), or any other wired transmission protocol. Similarly, the classical computing component 110 may be configured to communicate via wireless external communication networks using any of a variety of protocols, such as general packet radio service (GPRS), Universal Mobile Telecommunications System (UMTS), Code Division Multiple Access 2000 (CDMA2000), CDMA2000 1× (1×RTT), Wideband Code Division Multiple Access (WCDMA), Global System for Mobile Communications (GSM), Enhanced Data rates for GSM Evolution (EDGE), Time Division-Synchronous Code Division Multiple Access (TD-SCDMA), Long Term Evolution (LTE), Evolved Universal Terrestrial Radio Access Network (E-UTRAN), Evolution-Data Optimized (EVDO), High Speed Packet Access (HSPA), High-Speed Downlink Packet Access (HSDPA), IEEE 802.11 (Wi-Fi), Wi-Fi Direct, 802.16 (WiMAX), ultra wideband (UWB), infrared (IR) protocols, near field communication (NFC) protocols, Wibree, Bluetooth protocols, wireless universal serial bus (USB) protocols, and/or any other wireless protocol. The classical computing component 110 may use such protocols and standards to communicate using Border Gateway Protocol (BGP), Dynamic Host Configuration Protocol (DHCP), Domain Name System (DNS), File Transfer Protocol (FTP), Hypertext Transfer Protocol (HTTP), HTTP over TLS/SSL/Secure, Internet Message Access Protocol (IMAP), Network Time Protocol (NTP), Simple Mail Transfer Protocol (SMTP), Telnet, Transport Layer Security (TLS), Secure Sockets Layer (SSL), Internet Protocol (IP), Transmission Control Protocol (TCP), User Datagram Protocol (UDP), Datagram Congestion Control Protocol (DCCP), Stream Control Transmission Protocol (SCTP), HyperText Markup Language (HTML), and/or the like.

Via these communication standards and protocols, the classical computing component 110 can communicate with various other entities using concepts such as Unstructured Supplementary Service information/data (USSD), Short Message Service (SMS), Multimedia Messaging Service (MMS), Dual-Tone Multi-Frequency Signaling (DTMF), and/or Subscriber Identity Module Dialer (SIM dialer). The classical computing component 110 can also download changes, add-ons, and updates, for instance, to its firmware, software (e.g., including executable instructions, applications, program modules), and operating system.

In various embodiments, the classical computing component 110 may comprise a network interface 620 for interfacing and/or communicating with the controller 132, for example. For example, the classical computing component 110 may comprise a network interface 620 for providing qubit constraint information, executable instructions, command sets, and/or the like for receipt by the controller 132 and/or receiving output and/or the result of a processing the output (e.g., measured values corresponding to the active orbitals) provided by the quantum computing component 130. In various embodiments, the classical computing component 110 and the controller 132 may communicate via a direct wired and/or wireless connection and/or via one or more wired and/or wireless networks 120.

In various embodiments, the processing elements 608 may comprise one or more processing devices such as programmable logic devices (CPLDs), microprocessors, coprocessing entities, application-specific instruction-set processors (ASIPs), integrated circuits, application specific integrated circuits (ASICs), field programmable gate arrays (FPGAs), programmable logic arrays (PLAs), hardware accelerators, graphics processing units (GPUs), central processing units (CPUs), other processing devices and/or circuitry, and/or the like. The term circuitry may refer to an entirely hardware embodiment or a combination of hardware and computer program products.

The classical computing component 110 may also comprise a user interface device comprising one or more user input/output interfaces (e.g., a display 616 and/or speaker/speaker driver coupled to a processing element 608 and a touch screen, keyboard, mouse, and/or microphone coupled to a processing element 608). For instance, the user output interface may be configured to provide an application, browser, user interface, interface, dashboard, screen, webpage, page, and/or similar words used herein interchangeably executing on and/or accessible via the computing entity 10 to cause display or audible presentation of information/data and for interaction therewith via one or more user input interfaces. The user input interface can comprise any of a number of devices allowing the computing entity 10 to receive data, such as a keypad 618 (hard or soft), a touch display, mouse, voice/speech or motion interfaces, scanners, readers, or other input device. In embodiments including a keypad 618, the keypad 618 can include (or cause display of) the conventional numeric (0-9) and related keys (#, *), and other keys used for operating the classical computing component 110 and may include a full set of alphabetic keys or set of keys that may be activated to provide a full set of alphanumeric keys. In addition to providing input, the user input interface can be used, for example, to activate or deactivate certain functions, such as screen savers and/or sleep modes. Through such inputs the classical computing component 110 can collect information/data, user interaction/input, and/or the like.

The classical computing component 110 can also include volatile storage or memory 622 and/or non-volatile storage or memory 624, which can be embedded and/or may be removable. For instance, the non-volatile memory may be ROM, PROM, EPROM, EEPROM, flash memory, MMCs, SD memory cards, Memory Sticks, CBRAM, PRAM, FeRAM, RRAM, SONOS, racetrack memory, and/or the like. The volatile memory may be RAM, DRAM, SRAM, FPM DRAM, EDO DRAM, SDRAM, DDR SDRAM, DDR2 SDRAM, DDR3 SDRAM, RDRAM, RIMM, DIMM, SIMM, VRAM, cache memory, register memory, and/or the like. The volatile and non-volatile storage or memory can store databases, database instances, database management system entities, data, applications, programs, program modules, scripts, source code, object code, byte code, compiled code, interpreted code, machine code, executable instructions, and/or the like to implement the functions of the classical computing component 110.

### CONCLUSION

Many modifications and other embodiments of the invention set forth herein will come to mind to one skilled in the art to which the invention pertains having the benefit of the teachings presented in the foregoing descriptions and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

1. A method for simulating a chemical system using a hybrid quantum-classical computing system, the method comprising:
obtaining, by a classical computing component of the hybrid quantum-classical computing system, an indication of a chemical system;
evaluating, by the classical computing component, class selection metrics based at least in part on a structure of the chemical system;
determining, by the classical computing component, whether the class selection metrics satisfy one or more class selection criteria;
responsive to determining that class selection metrics do not satisfy the one or more class selection criteria, performing, by the classical computing component, at least one of (a) providing a notification that the class selection criteria are not satisfied by the chemical system or (b) performing a classical approximation to generate a model of the chemical system that represents at least one of: a structural characteristic of the chemical system, a chemical interaction characteristic of the chemical system, or a response characteristic;
responsive to determining that the class selection metrics do satisfy the one or more class selection criteria:
providing, by the classical computing component, qubit constraint information regarding an active-space electronic Hamiltonian for the chemical system to a quantum computing component of the hybrid quantum-classical computing system;
receiving, by the classical computing component, measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and
utilizing, by the classical computing component, the measured values to generate the model of the chemical system that represents at least one of: a structural characteristic of the chemical system, a chemical interaction characteristic of the chemical system, or a response characteristic.

2. The method of claim 1, wherein evaluating the class selection metrics comprises performing a multireference diagnostic of the chemical system to determine a multireference diagnostic number corresponding to the chemical system and determining whether the class selection metrics satisfy the one or more class selection criteria comprises determining whether the multireference diagnostic number corresponding to the chemical system is greater than a threshold strength.

3. The method of claim 2, wherein performing the multireference diagnostic of the chemical system comprises determining a classical approximation of a multireference wave function of the chemical system and determining a degree of electron correlation of the chemical system based at least in part on the classical approximation of the multireference wave function.

4. The method of claim 1, further comprising causing, by the classical computing component, at least one of (a) display of a graphical representation of at least a portion of the model of the chemical system or (b) generation and storage in a classical memory of a file comprising one or more parameters of the model of the chemical system.

5. The method of claim 1, further comprising, responsive to determining that the one or more class selection criteria are satisfied:
evaluating, by the classical computing entity, type selection metrics based on one or more of (a) at least a portion of a structure of the chemical system, (b) information about a quantum computing component of the hybrid quantum-classical computing system, or (c) user input; and
selecting a hybrid approximation based on one or more type selection criteria and the type selection metrics, wherein the classical computing component uses the hybrid approximation to generate the model of the chemical system utilizing the measured values.

6. The method of claim 5, wherein the second selection criteria are evaluated at least in part based on a number of active electrons of the chemical system, a number of active orbitals of the chemical system, or one or more symmetries of the chemical system.

7. The method of claim 5, wherein the second selection criteria are evaluated at least in part based on a number of qubits of the quantum computing component, available run time of the quantum computing component, or a noise profile of one or more functions of the quantum computing component.

8. The method of claim 5, wherein the hybrid approximation is one of a second-order N-electron valence state perturbation theory (NEVPT2) approximation or an AC0 approximation.

9. The method of claim 5, wherein the measured values that are measured by the quantum computing component are determined based on the hybrid approximation.

10. The method of claim 1, wherein the measured values comprise at least one of an expectation value of the active-space Hamiltonian or at least one reduced density matrix (RDM).

11. The method of claim 10, wherein the at least one RDM comprises at least one of a one particle RDM (1-RDM), a two particle RDM (2-RDM), a three particle RDM (3-RDM), or a four particle RDM (4-RDM).

12. The method of claim 1, further comprising generating the qubit constraint information, wherein generating the qubit constrain information comprises:
determining, by the classical computing component, fermionic constraint information regarding an active-space electronic Hamiltonian defined in an active space of two or more active orbitals of the chemical system;
translating, by the classical computing component, the fermionic constraint information regarding the active-space electronic Hamiltonian into a qubit basis to generate qubit constraint information regarding the active-space electronic Hamiltonian.

13. The method of claim 12, wherein the fermionic constraint information regarding the active-space electronic Hamiltonian defined in the active space of two or more active orbitals of the chemical system is performed as part of evaluating the class selection metrics.

14. The method of claim 1, further comprising:
performing, by the quantum computing component, state preparation of a plurality of qubits based at least in part on the qubit constraint information regarding the active-space electronic Hamiltonian; and
performing, by the quantum computing component, one or more measurement operations to determine the measured values based on quantum states of at least a portion of the plurality of qubits.

15. A method for simulating a chemical system using a hybrid quantum-classical computing system, the method comprising:
obtaining, by a classical computing component of the hybrid quantum-classical computing system, an indication of a chemical system;
evaluating, by the classical computing component, selection metrics based on one or more of (a) at least a portion of an atomic structure of the chemical system, (b) information about a quantum computing component of the hybrid quantum-classical computing system, or (c) user input;
selecting a hybrid approximation based on one or more type selection criteria and the selection metrics;
providing, by the classical computing component, qubit constraint information regarding an active-space electronic Hamiltonian for the chemical system to a quantum computing component of the hybrid quantum-classical computing system;
receiving, by the classical computing component, measured values (a) corresponding to expectation values of quantum operators acting on quantum states of at least a portion of a plurality of qubits of the quantum computing component and (b) representative of the expectation values of quantum operators acting on eigenstates of the active-space electronic Hamiltonian; and
utilizing, by the classical computing component, the measured values and the hybrid approximation to generate a model of the chemical system that represents at least one of: a structural characteristic of the chemical system, a chemical interaction characteristic of the chemical system, or a response characteristic.

16. The method of claim 15, wherein the second selection criteria are evaluated at least in part based on a number of active electrons of the chemical system, a number of active orbitals of the chemical system, or one or more symmetries of the chemical system.

17. The method of claim 15, wherein the second selection criteria are evaluated at least in part based on a number of qubits of the quantum computing component, available run time of the quantum computing component, or a noise profile of one or more functions of the quantum computing component.

18. The method of claim 15, wherein the hybrid approximation is one of a second-order N-electron valence state perturbation theory (NEVPT2) approximation or an AC0 approximation.

19. The method of claim 15, wherein the measured values that are measured by the quantum computing component are determined based on the hybrid approximation.

20. The method of claim 15, wherein the measured values comprise at least one of an expectation value of the active-space Hamiltonian or at least one reduced density matrix (RDM).

21-22. (canceled)
